(19) United States
(12) Patent Application Publication
(10) Pub. No.: US 2019/0160049 A1
(43) Pub. Date: May 30, 2019

(54) 2-OXO-1,2-DIHYDROPYRIDINE-3-CARBOXAMIDE COMPOUNDS AND THEIR USE AS DUAL INHIBITORS OF PDK1/AURA

(71) Applicant: INTERNATIONAL SOCIETY FOR DRUG DEVELOPMENT S.R.L., Milan (IT)

(72) Inventors: Simona SESTITO, Chiaravalle Centrale (IT); Simona DANIELE, Pisa (IT); Claudia MARTINI, Pisa (IT); Simona RAPPOSELLI, Capannori (IT); Guido PURICELLI, Milan (IT)

(21) Appl. No.: 16/308,690

(22) PCT Filed: Jun. 8, 2017
(86) PCT No.: PCT/EP2017/063950

§ 371 Date: Dec. 10, 2018

(30) Foreign Application Priority Data
Jun. 10, 2016 (IT) 102016000059838

(51) Int. Cl. A61K 31/4439; A61K 31/4375; A61P 35/00; A61K 31/55; C07D 401/14; C07D 471/04

(52) U.S. Cl. CPC A61K 31/4439; A61K 31/4375; C07D 471/04; A61K 31/55; C07D 401/14; A61P 35/00

(57) ABSTRACT

The present invention concern a 2-oxo-1,2-dihydropyridine-3-carboxamide compound of Formula (I) in the treatment of pathologies which require a dual inhibitor of PDK1/AurA enzymes such as for instance tumours, particularly glioblastoma.

### FIELD OF INVENTION

The invention concerns 2-oxo-1,2-dihydropyridine-3-carboxamide compounds and their use as dual inhibitors and/or modulators of PDK1/AurA.

### STATE OF THE ART

The PI3K/PDK1/Akt signaling axis is centrally involved in inhibition of apoptosis and stimulation of cell proliferation and it has been estimated that at least 50% of all cancer types are related to deregulation of this signaling pathway.

Due to its key function as regulator of cell survival and metabolism, the dysregulation of this pathway is manifested in several human pathologies including cancers and neurodegenerative diseases.

Phosphoinositide-dependent kinase (PDK1) acts as one of the main mediators of the pathway. PDK1 is a serine/threonine protein kinase that plays a key role in regulating cell growth, proliferation, and survival through both Akt-dependent and Akt-independent mechanisms. The Akt-dependent pathway is characterized by the implication of downstream proteins like mTOR, Ras and GSK, all controlled by Akt. The Akt-independent signal acts via PLCγ1, a phospholipase implicated in metastasis.

The phosphorylation and, therefore, activation of multiple substrates that seem to be constitutively active in tumor tissue (such as AKT, S6K, SGK, RSK and PKC isoforms) may explain the influence of this kinase on a variety of cellular processes including proliferation, migration and survival.

For these reasons, PDK1 also known as “master kinase” of the AGC kinases has attracted considerable interest as an anticancer drug target. However, although there have been done huge efforts in discovering specific molecules targeting PI3K and Akt, PDK1 has been rather overlooked. Recently the increasing interest in this kinase prompted many research groups to work in this direction, thus publishing and patenting several series of molecules able to inhibit this important node of the PI3K/PDK1/Akt.

PDK1 plays a pleiotropic role in growth and development. Recent findings revealed that elevated activation of PDK1 induces tumorigenesis by enhancing cell proliferation and inhibiting apoptosis. In addition, increasing evidence show that PDK1 plays a pivotal role in cell migration and metastasis. Its role in these processes was proved in different cell types and organisms including endothelial cells, smooth muscle cells, T lymphocytes, neutrophils and several tumour cell lines such as breast, glioblastoma (Signore, M., et al., Combined PDK1 and CHK1 inhibition is required to kill glioblastoma stem-like cells in vitro and in vivo. Cell death & disease, 2014. 5(5): p. e1223) and pancreatic (Ferro R. et al Emerging role of the KRAS-PDK1 axis in pancreatic cancer. World J Gastroenterol. 2014, 20(31):10752-7) cancers.

Unfortunately, nowadays no selective PDK1 inhibitor has entered the clinic, making the “master kinase” of AGC family a target not yet exploited in the clinic.

Noteworthy, many other key signaling pathways interact with PI3K/PDK1/Akt, including Notch, MNK, Syk, MAPK, and Aurora kinases. For instance, aberrations of Aurora A (Aur-A or AurA), such as overexpression, are associated with many type of cancers including GBM (Lee P. Y. et al, The Aurora kinases inhibitor VE-465 is a novel treatment for glioblastoma multiforme, Oncology, 84 (2013) 326-335; De Bacco F. et al. MET inhibition overcomes radiation resistance of glioblastoma stem-like cells, 8 (2016) 550-568.) Between the several activities of Aur-A are included the regulation of mitotic entry, centrosome maturation and spindle formation. Since mitosis is a fine process, this protein was largely investigated as potential anticancer target [M. Malumbres, I. Pérez de Castro, Aurora kinase A inhibitors: promising agents in antitumoral therapy, Expert opinion on therapeutic targets, 18 (2014) 1377-1393]. Precisely Aurora A is involved in cellular pro-oncogenic signalling through both its mitotic and non-mitotic function: it is essential for DNA damage induced checkpoint recovery [L. Macůrek, A. Lindqvist, D. Lim, M. A. Lampson, R. Klompmaker, R. Freire, C. Clouin, S. S. Taylor, M. B. Yaffe, R. H. Medema, Polo-like kinase-1 is activated by aurora A to promote checkpoint recovery, Nature, 455 (2008) 119-123], regulates the activity of the “guardian of genome” p53, but also modulate the PI3K/Akt/PDK1 activity [J.-e. Yao, M. Yan, Z. Guan, C.-b. Pan, L.-p. Xia, C.-x. Li, L.-h. Wang, Z.-j. Long, Y. Zhao, M.-w. Li, Aurora-A down-regulates IkappaBα via Akt activation and interacts with insulin-like growth factor-1 induced phosphatidylinositol 3-kinase pathway for cancer cell survival, Molecular cancer, 8 (2009)]. In glioblastoma (GBM), Aurora A was related to aggressive behavior [W. F. Zeng, K. Navaratne, R. A. Prayson, R. J. Weil, Aurora B expression correlates with aggressive behaviour in glioblastoma multiforme, Journal of clinical pathology, 60 (2007) 218-221]; more interestingly Alisertib, a selective Aurora A inhibitor already in clinical trials, showed to potently inhibit proliferation of GBM neurosphere tumor stem-like cells, also potentiating the effects of classic GBM therapy, such as temozolomide and ionizing radiation [X. Hong, J. P. O'Donnell, C. R. Salazar, J. R. Van Brocklyn, K. D. Barnett, D. K. Pearl, J. A. Ecsedy, S. L. Brown, T. Mikkelsen, N. L. Lehman, The selective Aurora-A kinase inhibitor MLN8237 (alisertib) potently inhibits proliferation of glioblastoma neurosphere tumor stem-like cells and potentiates the effects of temozolomide and ionizing radiation, Cancer chemotherapy and pharmacology, 73 (2014) 983-990].

Therefore, the inventors pointed at aiming the development of new multitarget agents with the aim of hit two specific kinases (PDK1 and Aurora A) which are nodal points for aggressiveness, chemoresistance, recurrence and metastasis formation in cancer.

It is an object of the invention hence to provide small molecules capable to disrupt the Aurora A/PDK1 axis to achieve different goals: the inhibition of cell proliferation, the induction of apoptosis, the delaying of cell migration and metastasis formation and, last but not least, the induction of differentiation and senescence in Glioblastoma Stem Cells (GSCs), the hard core of glioblastoma.

### SUMMARY OF THE INVENTION

The above object has been achieved by a 2-oxo-1,2-dihydropyridine-3-carboxamide compound of Formula (I)

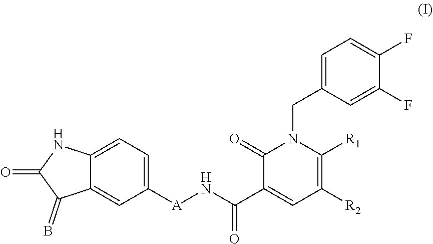

or a pharmaceutical salt thereof

wherein

B is CH-D, where D is imidazolyl; and

A is selected from the group consisting of (—NH—CO—CH₂—), (—NH—CO—CH₂—CH₂), (—NH—CO—CH(Ph)-) and (—NH—CO—CH₂—CH₂—CH₂)

R₁ is H or CH₃, R₂ is H or Br or R₁ and R₂ taken together form the group —(N═CH—CH═CH)—

for use in the treatment of pathologies requiring the use of a dual inhibitor of PDK1/AurA enzymes,

with the proviso that

when A is selected from (—NH—CO—CH₂—CH₂) and (—NH—CO—CH₂—CH₂—CH₂), then R₁ and R₂ are H.

In another aspect the invention concerns a new 2-oxo-1,2-dihydropyridine-3-carboxamide compound of Formula (I)

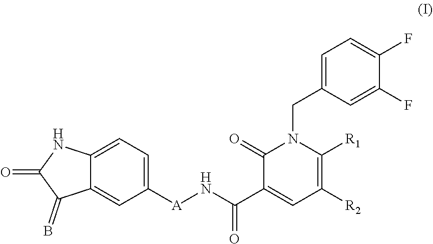

or a pharmaceutical salt thereof

wherein

B is CH-D, where D is imidazolyl; and

A is selected from the group consisting of (—NH—CO—CH₂—), (—NH—CO—CH(Ph)-) and (—NH—CO—CH₂—CH₂—CH₂)

R₁ is H or CH₃, R₂ is H or Br or R₁ and R₂ together represent the group —(N═CH—CH═C)—

with the proviso that

when A is (—NH—CO—CH₂—CH₂—CH₂) then R₁ and R₂ are H and

when A is (—NH—CO—CH₂—) or (—NH—CO—CH(Ph)-), then R₁ and R₂ are not simultaneously H.

In another aspect the invention concerns a compound of Formula (I) for use as a medicament.

In a further aspect the invention concerns a pharmaceutical composition comprising a compound of Formula (I) and a pharmaceutically acceptable carrier.

In a still further aspect the invention concerns a compound of Formula (I) for use in the treatment of pathologies requiring the use of a dual inhibitor of PDK1/AurA. The pathologies that require a dual inhibitor of PDK1/AurA enzymes include a broad range of human solid tumors, such as primary colorectal carcinoma, gliomas, breast, ovarian, pancreatic cancer and hematologic malignancies such as multiple myeloma, Non-Hodgkin lymphoma, and chronic lymphocytic leukemia. PDK1 and AurA enzymes are also involved in neurodegenerative diseases such as Alzheimer's disease, Parkinson's disease and Huntington's disease and cardiovascular diseases such as diabetes. Preferably such pathology is a cancer, more preferably glioblastoma (GBM).

In this invention a compound of Formula (I) may exist as R and S enantiomers and as racemic mixture. This invention includes in its scope of protection all the possible isomers and racemic mixtures. Wherever should be present further symmetry centers, this invention includes all the possible diastereoisomers and relative mixtures as well.

In this invention a combination of a PDK1 and AurA inhibitors has been evaluated as a cutting-edge therapy to treat GBM and surprisingly the inventors found out that the pharmaceutical combination comprising at least one PDK1 inhibitor and at least one AurA inhibitor was capable to treat tumors with respect to the respective inhibitors used alone, specifically in case of glioblastoma.

Therefore in another aspect the invention concerns a pharmaceutical combination comprising at least one PDK1 inhibitor and at least one AurA inhibitor. Preferably the at least one PDK1 inhibitor is MP7 (1-(3,4-difluorobenzyl)-2-oxo-N-{(1R)-2-[(2-oxo-2,3-dihydro-1H-benzimidazol-5-yl)oxy]-1-phenylethyl}-1,2-dihydropyridine-3-carboxamide) and the at least one AurA inhibitor is Alisertib_4-{[9-Chloro-7-(2-fluoro-6-methoxyphenyl)-5H-pyrimido[5,4-d][2]benzazepin-2-yl]amino}-2-methoxybenzoic acid).

More surprisingly and as it will be clear below the inventors found out that the 2-oxo-1,2-dihydropyridine-3-carboxamide compound of Formula (I) were better dual inhibitor than the above pharmaceutical combination.

### DESCRIPTION OF THE FIGURES

FIG. 1 illustrates the effects of SA16 on U87MG cell proliferation of example 3 (a). U87MG cells were treated in complete medium with different concentrations of SA16 (1 nM-100 μM) for 72 h. At the end of treatment, cell proliferation was measured using the MTS assay. The data are expressed as a percentage with respect to that of untreated cells (control), which was set to 100%, and are the mean values±SEM of three independent experiments, each performed in duplicate. The significance of the differences was determined with a one-way ANOVA with Bonferroni post-test: * p<0.05, ** p<0.01, *** p<0.001 vs. control cells;

FIG. 2 illustrates the combined inhibition of PDK1 and Aurora A on U87MG cell proliferation of example 3a. U87MG cells were treated in complete medium with different concentrations of MP7, in the presence or absence of Alisertib, for 72 h. At the end of treatment, cell proliferation was measured using the MTS assay. The data are expressed as a percentage with respect to that of untreated cells (control), which was set to 100%, and are the mean values±SEM of three independent experiments, each performed in duplicate. The significance of the differences was determined with a one-way ANOVA with Bonferroni post-test: * p<0.05, *** p<0.001 vs. control cells; ## p<0.01, ### p<0.001 vs. cells treated with MP7 alone; §§ p<0.01, § § § p<0.001 vs. cells treated with Alisertib alone;

FIG. 3 illustrates the effects of SA16 on CSC proliferation of example 3b. CSCs were treated in complete neurosphere medium with different concentrations of SA16 (1 nM-100 μM) for seven days. At the end of treatment, cell proliferation was measured using the MTS assay. The data are expressed as a percentage with respect to that of untreated cells (control), which was set to 100%, and are the mean values±SEM of three independent experiments, each performed in duplicate. IC₅₀ value after seven days of treatment were calculated from sigmoid dose-response curve;

FIG. 4 illustrates combined inhibition of PDK1 and Aurora A on CSCs proliferation of example 3b. CSCs were treated in complete medium with different concentrations of MP7, in the presence or absence of Alisertib, for seven days. At the end of treatment, cell proliferation was measured using the MTS assay.

The data are expressed as a percentage with respect to that of untreated cells (control), which was set to 100%, and are the mean values±SEM of three independent experiments, each performed in duplicate. The significance of the differences was determined with a one-way ANOVA with Bonferroni post-test: * p<0.05, *** p<0.001 vs. control cells; ## p<0.01, ### p<0.001 vs. cells treated with MP7 alone; §§ p<0.01, § § § p<0.001 vs. cells treated with Alisertib alone;

FIG. 5 illustrates the effects of SA16 on sphere-derived cell morphology of Example 3c. CSCs were treated for seven days with complete NSC medium containing DMSO (control), or SA16 (10 nM, 1 μM, 10 μM) (A) Representative cell micrographs of control and SA16 (10 μM) after seven days of treatment are shown. The area of the culture plates occupied by the spheres (B) and the length of cellular processes (C) were scored after seven days of treatment. The counts represent the mean values±SEM of two independent experiments. The significance of differences was determined with a one-way ANOVA with Bonferroni post-test: * p<0.05, ** p<0.01, *** p<0.001 vs. control cells.

FIG. 6 illustrates the effects of SA16 on CSC differentiation of example 3c. CSCs were treated for seven days with complete NSC medium containing DMSO (control), or SA16 (10 μM) and the relative mRNA quantification of the stem cell marker nestin, the neuronal marker MAP, and of the glial marker GFAP was performed by RT-PCR. The data are expressed as the fold change vs. the levels of the control and are the mean values±SEM of three different experiments. The significance of the differences was determined with a one-way ANOVA with Bonferroni post-test: ** p<0.01, *** p<0.001 vs. control.

FIG. 7 illustrates the effects of MP7, Alisertib and of their combined treatment on sphere-derived cell morphology of example 3c. CSCs were treated for seven days with complete NSC medium containing DMSO (control), MP7 and/or alisertib (A) Representative cell micrographs after seven days of treatment are shown. The area of the culture plates occupied by the spheres (B) and the length of cellular processes (C) were scored after seven days of treatment. The counts represent the mean values±SEM of two independent experiments. The significance of differences was determined with a one-way ANOVA with Bonferroni post-test: * p<0.05, *** p<0.001 vs. control cells; ## p<0.01 vs. cells treated with MP7 alone; § p<0.05 vs. cells treated with Alisertib alone.

### DETAILED DESCRIPTION OF THE INVENTION

The invention hence concerns a 2-oxo-1,2-dihydropyridine-3-carboxamide compound of Formula (I)

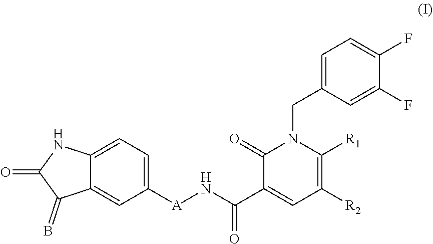

or a pharmaceutical salt thereof

wherein

B is CH-D, where D is imidazolyl; and

A is selected from the group consisting of (—NH—CO—CH₂—), (—NH—CO—CH₂—CH₂), (—NH—CO—CH(Ph)-) and (—NH—CO—CH₂—CH₂—CH₂)

R₁ is H or CH₃, R₂ is H or Br or R₁ and R₂ taken together form the group —(N═CH—CH═CH)—

for use in the treatment of pathologies requiring the use of a dual inhibitor of PDK1/AurA enzymes,

with the proviso that

when A is selected from (—NH—CO—CH₂—CH₂) and (—NH—CO—CH₂—CH₂—CH₂), then R₁ and R₂ are H.

A is selected from the group consisting of (—NH—CO—CH₂—), (—NH—CO—CH₂—CH₂), (—NH—CO—CH(Ph)-) and (—NH—CO—CH₂—CH₂—CH₂). Preferably A is (—NH—CO—CH₂—) or (—NH—CO—CH(Ph)-), more preferably A is (—NH—CO—CH(Ph)-).

In the preferred embodiment wherein A is (—NH—CO—CH(Ph)-), R₁ is preferably CH₃ and R₂ is preferably H.

Alternatively, In the preferred embodiment wherein A is (—NH—CO—CH(Ph)-), R₁ is preferably CH₃ and R₂ is preferably Br.

In a further preferred embodiment A is (—NH—CO—CH(Ph)-), R₁ and R₂ are H.

In another preferred embodiment A is (—NH—CO—CH₂—), R₁ is preferably CH₃ and R₂ is preferably H.

Alternatively, In the preferred embodiment wherein A is (—NH—CO—CH₂—), R1 is preferably CH₃ and R2 is preferably Br.

In another preferred embodiment A is (—NH—CO—CH₂—), R1 and R2 form the group —(N═CH—CH═CH)—

In a further preferred embodiment A is (—NH—CO—CH₂—), R1 and R2 are H.

In another preferred embodiment A is (—NH—CO—CH₂—CH₂).

B is CH-D, where D is imidazolyl, preferably 1H-imidazol-5-yl or 1H-imidazol-2-yl, more preferably 1H-imidazol-5-yl.

In a more preferred embodiment A is (—NH—CO—CH₂—) or (—NH—CO—CH(Ph)-) and B is preferably 1H-imidazol-5-yl.

The preferred compound for the use as dual inhibitor of PDK1/AurA enzymes is one of the compounds reported in the Table below.

| Structure | IUPAC name |
|---|---|
| 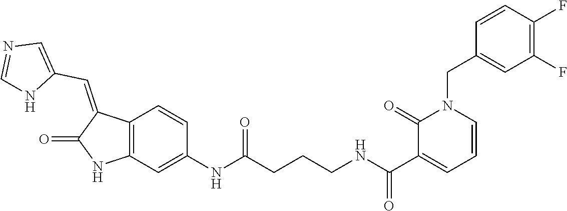 [SMILES: O=C(CCCNC(=O)c1cccn(Cc2ccc(F)c(F)c2)c1=O)Nc1ccc2c(c1)NC(=O)/C2=C\c1cnc[nH]1] | DF8 (Z)-N-(4-((3-((1H-imidazol-5- yl)methylene)-2-oxoindolin-6- yl)amino)-4-oxobutyl)-1-(3,4- difluorobenzyl)-2-oxo-1,2- dihydropyridine-3-carboxamide |
| 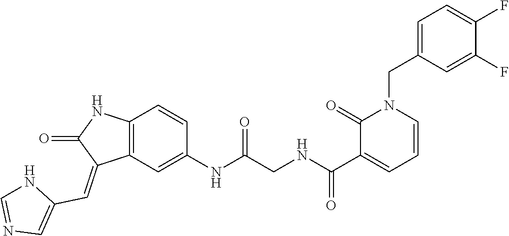 [SMILES: O=C(CNC(=O)c1cccn(Cc2ccc(F)c(F)c2)c1=O)Nc1ccc2c(c1)/C(=C/c1cnc[nH]1)C(=O)N2] | IB35 (Z)-N-(2-((3-((1H-imidazol-5- yl)methylene)-2-oxoindolin-5- yl)amino)-2-oxoethyl)-1-(3,4- difluorobenzyl)-2-oxo-1,2-dihydro pyridine-3-carboxamide |
| 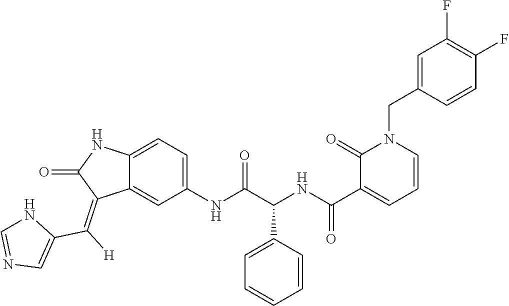 [SMILES: O=C1Nc2ccc(NC(=O)[C@H](NC(=O)c3cccn(Cc4ccc(F)c(F)c4)c3=O)c3ccccc3)cc2/C1=C/c1cnc[nH]1] | SA16 (Z)-(R)-N-(2-((3-((1H-imidazol-5- yl)methylene)-2-oxoindolin-5- yl)amino)-2-oxo-1-phenylethyl)-1- (3,4-difluorobenzyl)-2-oxo-1,2- dihydropyridine-3-carboxamide |
| 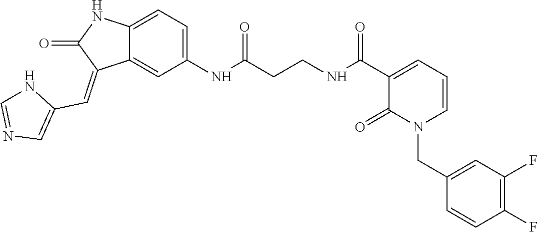 [SMILES: O=C(CCNC(=O)c1cccn(Cc2ccc(F)c(F)c2)c1=O)Nc1ccc2c(c1)/C(=C/c1cnc[nH]1)C(=O)N2] | DD21 (Z)-N-(3-((3-((1H-imidazol-5- yl)methylene)-2-oxoindolin-5- yl)amino)-3-oxopropyl)-1-(3,4- difluorobenzyl)-2-oxo-1,2- dihydropyridine-3-carboxamide |
| 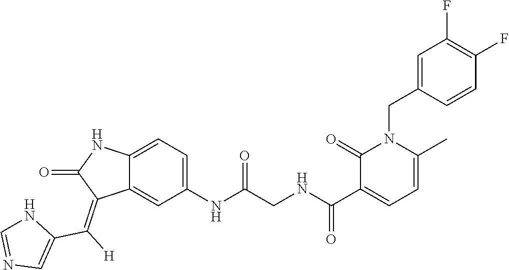 [SMILES: Cc1ccc(C(=O)NCC(=O)Nc2ccc3c(c2)/C(=C/c2cnc[nH]2)C(=O)N3)c(=O)n1Cc1ccc(F)c(F)c1] | SST200: (Z)-N-(2-((3-((1H-imidazol-5- yl)methylene)-2-oxoindolin-5- yl)amino)-2-oxoethyl)-1-(3,4- difluorobenzyl)-6-methyl-2- oxo-1,2-dihydropyridine-3- carboxamide |
| 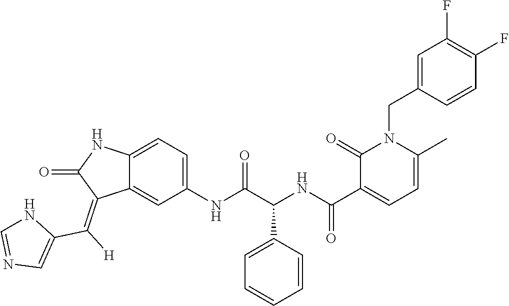 [SMILES: Cc1ccc(C(=O)N[C@@H](C(=O)Nc2ccc3c(c2)/C(=C/c2cnc[nH]2)C(=O)N3)c2ccccc2)c(=O)n1Cc1ccc(F)c(F)c1] | VI8 (R,Z)-N-(2-((3-((1H-imidazol-5- yl)methylene)-2-oxoindolin-5- yl)amino)-2-oxo-1-phenylethyl)- 1-(3,4-difluorobenzyl)-6-methyl- 2-oxo-1,2-dihydropyridine- 3-carboxamide |
| 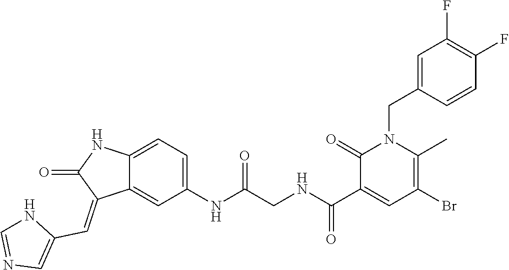 [SMILES: Cc1c(Br)cc(C(=O)NCC(=O)Nc2ccc3c(c2)/C(=C/c2cnc[nH]2)C(=O)N3)c(=O)n1Cc1ccc(F)c(F)c1] | VI23 (Z)-N-(2-((3-((1H-imidazol-5- yl)methylene)-2-oxoindolin-5- yl)amino)-2-oxoethyl)-5-bromo- 1-(3,4-difluorobenzyl)-6-methyl- 2-oxo-1,2-dihydropyridine- 3-carboxamide |
| 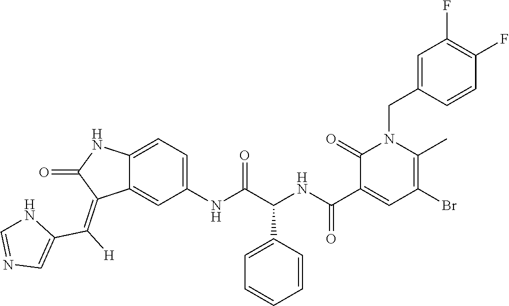 [SMILES: Cc1c(Br)cc(C(=O)N[C@@H](C(=O)Nc2ccc3c(c2)/C(=C/c2cnc[nH]2)C(=O)N3)c2ccccc2)c(=O)n1Cc1ccc(F)c(F)c1] | VI18 (R,Z)-N-(2-((3-((1H-imidazol- 5-yl)methylene)-2-oxoindolin- 5-yl)amino)-2-oxo-1-phenyl ethyl)-5-bromo-1-(3,4- difluorobenzyl)-6-methyl- 2-oxo-1,2-dihydropyridine- 3-carboxamide |
| 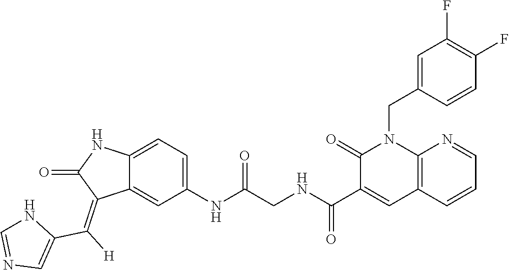 [SMILES: O=C(CNC(=O)c1cc2cccnc2n(Cc2ccc(F)c(F)c2)c1=O)Nc1ccc2c(c1)/C(=C/c1cnc[nH]1)C(=O)N2] | SST201 (Z)-N-(2-((3-((1H-imidazol-5- yl)methylene)-2-oxoindolin-5- yl)amino)-2-oxoethyl)-1-(3,4- difluorobenzyl)-2-oxo-1,2- dihydro-1,8-naphthyridine- 3-carboxamide |

More preferably the compound of Formula (I) is selected from the group (Z)—N-(4-((3-((1H-imidazol-5-yl)methylene)-2-oxoindolin-5-yl)amino)-4-oxobutyl)-1-(3,4-difluorobenzyl)-2-oxo-1,2-dihydropyridine-3-carboxamide(DF8), (Z)—N-(2-((3-((1H-imidazol-5-yl)methyl ene)-2-oxoindoin-5-yl)amino)-2-oxoethyl)-1-(3,4-difluorobenzyl)-2-oxo-1,2-dihydro pyridine-3-carboxamide(IB35), (Z)—(R)—N-(2-((3-((1H-imidazol-5-yl)methylene)-2-oxoindolin-5-yl)amino)-2-oxo-1-phenylethyl)-1-(3,4-difluorobenzyl)-2-oxo-1,2-dihydropyridine-3-carboxamide (SA16), (Z)—N-(2-((3-((1H-imidazol-5-yl)methylene)-2-oxoindolin-5-yl)amino)-2-oxoethyl)-1-(3,4-difluorobenzyl)-6-methyl-2-oxo-1,2-dihydropyridine-3-carboxamide (SST200), (R,Z)—N-(2-((3-((1H-imidazol-5-yl)methylene)-2-oxoindolin-5-yl)amino)-2-oxo-1-phenylethyl)-1-(3,4-difluorobenzyl)-6-methyl-2-oxo-1,2-dihydropyridine-3-carboxamide (VI8), and (R,Z)—N-(2-((3-((1H-imidazol-5-yl)methylene)-2-oxoindolin-5-yl)amino)-2-oxo-1-phenylethyl)-5-bromo-1-(3,4-difluorobenzyl)-6-methyl-2-oxo-1,2-dihydropyridine-3-carboxamide (VI18).

Still more preferably the compound is selected from the group consisting of (Z)—(R)—N-(2-((3-((1H-imidazol-5-yl)methylene)-2-oxoindolin-5-yl)amino)-2-oxo-1-phenylethyl)-1-(3,4-difluorobenzyl)-2-oxo-1,2-dihydropyridine-3-carboxamide (SA16), (Z)—N-(2-((3-((1H-imidazol-5-yl)methylene)-2-oxoindolin-5-yl)amino)-2-oxoethyl)-1-(3,4-difluorobenzyl)-6-methyl-2-oxo-1,2-dihydropyridine-3-carboxamide (SST200), (R,Z)—N-(2-((3-((1H-imidazol-5-yl)methylene)-2-oxoindolin-5-yl)amino)-2-oxo-1-phenylethyl)-1-(3,4-difluorobenzyl)-6-methyl-2-oxo-1,2-dihydropyridine-3-carboxamide (VI8) and (R,Z)—N-(2-((3-((1H-imidazol-5-yl)methylene)-2-oxoindolin-5-yl)amino)-2-oxo-1-phenylethyl)-5-bromo-1-(3,4-difluorobenzyl)-6-methyl-2-oxo-1,2-dihydropyridine-3-carboxamide (VI18).

In another aspect the invention relates a new 2-oxo-1,2-dihydropyridine-3-carboxamide compound of Formula (I)

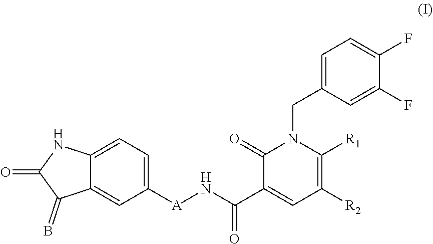

or a pharmaceutical salt thereof

wherein

B is CH-D, where D is imidazolyl; and

A is selected from the group consisting of (—NH—CO—CH₂—), (—NH—CO—CH(Ph)-) and (—NH—CO—CH₂—CH₂—CH₂)

R₁ is H or CH₃, R₂ is H or Br or R₁ and R₂ together represent the group —(N═CH—CH═C)—

with the proviso that

when A is (—NH—CO—CH₂—CH₂—CH₂) then R₁ and R₂ are H and

when A is (—NH—CO—CH₂—) or (—NH—CO—CH(Ph)-), then R₁ and R₂ are not simultaneously H.

A is (—NH—CO—CH₂—), (—NH—CO—CH(Ph)-) and (—NH—CO—CH₂—CH₂—CH₂). Preferably A is (—NH—CO—CH₂—CH₂—CH₂) or (—NH—CO—CH(Ph)-), still more preferably (—NH—CO—CH(Ph)-).

In the preferred embodiment wherein A is (—NH—CO—CH(Ph)-), R₁ is preferably CH₃ and R₂ preferably is H.

In the preferred embodiment wherein A is (—NH—CO—CH(Ph)-), R₁ is preferably CH₃ and R₂ is preferably Br.

B is CH-D, where D is imidazolyl, more preferably 1H-imidazol-5-yl.

In a preferred embodiment A is (—NH—CO—CH(Ph)-) and B is imidazolyl.

The preferred compound is one of the compounds reported in the Table below.

| Structure | IUPAC name |
|---|---|
| 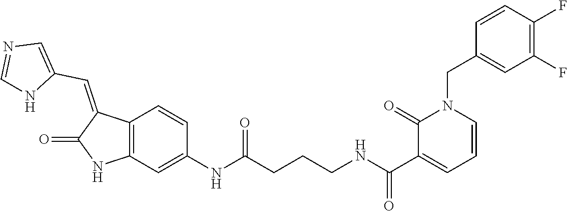 [SMILES: O=C(CCCNC(=O)c1cccn(Cc2ccc(F)c(F)c2)c1=O)Nc1ccc2c(c1)NC(=O)/C2=C\c1cnc[nH]1] | DF8 (Z)-N-(4-((3-((1H-imidazol-5- yl)methylene)-2-oxoindolin-6- yl)amino)-4-oxobutyl)-1-(3,4- difluorobenzyl)-2-oxo-1,2- dihydropyridine-3-carboxamide |
| 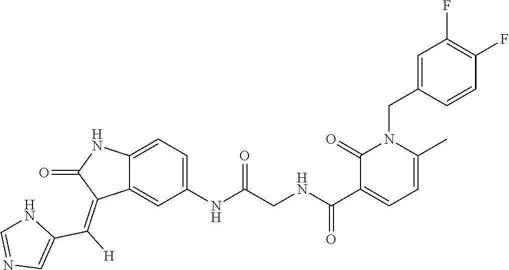 [SMILES: Cc1ccc(C(=O)NCC(=O)Nc2ccc3c(c2)/C(=C/c2cnc[nH]2)C(=O)N3)c(=O)n1Cc1ccc(F)c(F)c1] | SST200: (Z)-N-(2-((3-((1H-imidazol-5- yl)methylene)-2-oxoindolin-5- yl)amino)-2-oxoethyl)-1-(3,4- difluorobenzyl)-6-methyl-2- oxo-1,2-dihydropyridine-3- carboxamide |
| 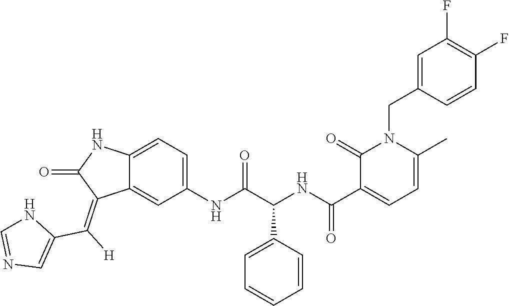 [SMILES: Cc1ccc(C(=O)N[C@@H](C(=O)Nc2ccc3c(c2)/C(=C/c2cnc[nH]2)C(=O)N3)c2ccccc2)c(=O)n1Cc1ccc(F)c(F)c1] | VI8 (R,Z)-N-(2-((3-((1H-imidazol-5- yl)methylene)-2-oxoindolin-5- yl)amino)-2-oxo-1-phenylethyl)- 1-(3,4-difluorobenzyl)-6-methyl- 2-oxo-1,2-dihydropyridine-3- carboxamide |
| 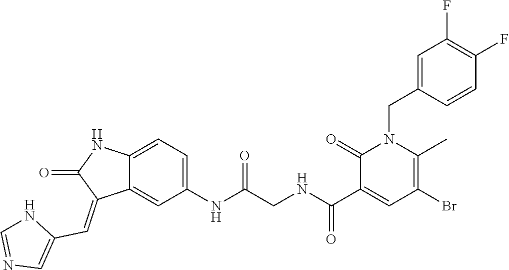 [SMILES: Cc1c(Br)cc(C(=O)NCC(=O)Nc2ccc3c(c2)/C(=C/c2cnc[nH]2)C(=O)N3)c(=O)n1Cc1ccc(F)c(F)c1] | VI23 (Z)-N-(2-((3-((1H-imidazol-5- yl)methylene)-2-oxoindolin-5- yl)amino)-2-oxoethyl)-5-bromo- 1-(3,4-difluorobenzyl)-6-methyl- 2-oxo-1,2-dihydropyridine- 3-carboxamide |
| 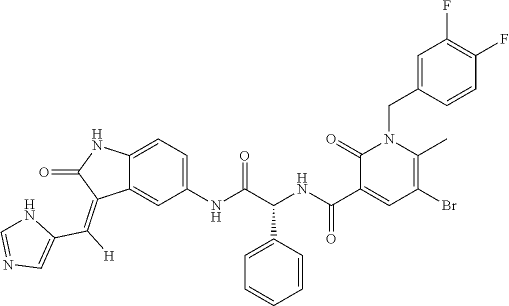 [SMILES: Cc1c(Br)cc(C(=O)N[C@@H](C(=O)Nc2ccc3c(c2)/C(=C/c2cnc[nH]2)C(=O)N3)c2ccccc2)c(=O)n1Cc1ccc(F)c(F)c1] | VI18 (R,Z)-N-(2-((3-((1H-imidazol-5- yl)methylene)-2-oxoindolin-5- yl)amino)-2-oxo-1-phenyl ethyl)-5-bromo-1-(3,4- difluorobenzyl)-6-methyl- 2-oxo-1,2-dihydropyridine- 3-carboxamide |
| 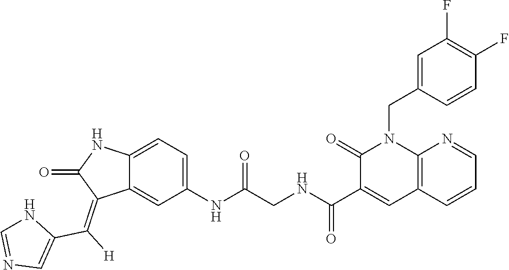 [SMILES: O=C(CNC(=O)c1cc2cccnc2n(Cc2ccc(F)c(F)c2)c1=O)Nc1ccc2c(c1)/C(=C/c1cnc[nH]1)C(=O)N2] | SST201 (Z)-N-(2-((3-((1H-imidazol-5- yl)methylene)-2-oxoindolin-5- yl)amino)-2-oxoethyl)-1-(3,4- difluorobenzyl)-2-oxo-1,2- dihydro-1,8-naphthyridine- 3-carboxamide |

More preferably the compound of Formula (I) is (Z)—N-(2-((3-((1H-imidazol-5-yl)methylene)-2-oxoindolin-5-yl)amino)-2-oxoethyl)-1-(3,4-difluorobenzyl)-6-methyl-2-oxo-1,2-dihydropyridine-3-carboxamide (SST200) or (R,Z)—N-(2-((3-((1H-imidazol-5-yl)methylene)-2-oxoindolin-5-yl)amino)-2-oxo-1-phenylethyl)-1-(3,4-difluorobenzyl)-6-methyl-2-oxo-1,2-dihydropyridine-3-carboxamide (VI8).

The compounds of the invention can be prepared by using processes, easy to scale-up and avoiding lengthy and expensive preparation steps thus obtaining high yield of a stable pharmaceutical grade compound as it will be evident from the experimental part of the present description.

The compounds of the invention of Formula (I) as such or a pharmaceutical salt thereof could be used in medicine in particular as dual inhibitor of PDK1/AurA enzymes.

In this invention a combination of a PDK1 and AurA inhibitors has been evaluated as a cutting-edge therapy to treat GBM and surprisingly the inventors found out that the pharmaceutical combination comprising at least one PDK1 inhibitor and at least one AurA inhibitor was capable to treat tumors with respect to the respective inhibitors used alone, specifically in case of glioblastoma.

Therefore in another aspect the invention concerns a pharmaceutical combination comprising at least one PDK1 inhibitor and at least one AurA inhibitor.

Preferably the at least one PDK1 inhibitor is MP7 (1-(3,4-difluorobenzyl)-2-oxo-N-{(1R)-2-[(2-oxo-2,3-dihydro-1H-benzimidazol-5-yl)oxy]-1-phenylethyl}-1,2-dihydropyridine-3-carboxamide) and the at least one AurA inhibitor is Alisertib_4-{[9-Chloro-7-(2-fluoro-6-methoxyphenyl)-5H-pyrimido[5,4-d][2]benzazepin-2-yl]amino}-2-methoxybenzoic acid).

The combined treatment of MP7 and Alisertib showed synergic/additive anti-proliferative effects as it will be evident in the experimental part.

In another aspect, the present invention provides a pharmaceutical composition comprising a compound of Formula (I) and a pharmaceutically acceptable excipient, for example a carrier. The pharmaceutical composition can also comprise a known PDK1 inhibitor compound and/or a known AurA inhibitor compound.

The compounds of the invention of Formula (I) can be administered alone or can be coadministered to the patient. Coadministration is meant to include simultaneous or sequential administration of the compounds individually or in combination (more than one compound). Thus, the preparation can also be combined, when desired, with other active substances, They could be used in combination with a pharmaceutically acceptable carrier and optionally with suitable excipients to obtain pharmaceutical compositions.

The term “pharmaceutically acceptable carrier” means solvents, carrier agents, diluting agents, and the like which are used in the administration of compounds of the invention.

Such pharmaceutical compositions can be administered by parenteral, oral, buccal, sublingual, nasal, rectal, topical or transdermal administration.

Compositions of the invention suitable for the oral administration will be conveniently discrete units such as tablets, capsules, cachet, powders or pellets or as liquid suspension.

The tablet can contain also suitable excipients routinely used in pharmaceutical field such as pre-gelatinized starch, microcrystalline cellulose, sodium glycolate starch, talc, lactose, magnesium stearate, sucrose, stearic acid, mannitol.

Compositions for parental administration may conveniently include sterile preparations.

Composition for topical administration may conveniently be formulated as creams, pastes, oils, ointments, emulsions, foams, gels, drops, spray solutions and transdermal patches.

The compounds of the invention can be used as a medicament in the treatment of pathologies which require a dual inhibitor of PDK1/AurA enzymes such as cancers, preferably in the treatment of glioblastoma (GBM).

The compounds of Formula (I) showed to inhibit both the PDK1 enzyme and AurA enzyme with IC50 values in the range of nM to μM as it will be evident from the experimental part of the description.

The ranking of IC50 value on recombinant PDK1/AurA reflected the affinity ranking towards glioblastoma cell lines, thus confirming that the antiproliferative activity is mediated by PDK1/AurA.

Advantageously the preferred compounds dually inhibited PDK1/AurA constitutive activity in glioblastoma cells and inhibited the Cancer Stem Cells (CSC) proliferation; as a result, the compounds of Formula (I) decreased cell viability, and triggered apoptosis. Moreover, the inhibition of cell viability was long-lasting. Also the combination MP7 and Alisertib decreased cell viability and triggered apoptosis. The combined treatment of MP7 and Alisertib showed synergic/additive anti-proliferative effects which is comparable to that obtained with SA16.

The effects of PDK1 and AurA inhibition were evaluated on GSCs isolated from U87MG cells. In the evaluation assay, PDK1/AurA inhibitors showed to promote CSCs toward a neuronal and a glial phenotype as well as the combination of MP7 and alisertib. The maximal effects of the co-treatment protocol were even lower than those obtained with the dual target compound SA16. Advantageously, the dual inhibition of PDK1 and AurA is a useful strategy to inhibit CSC proliferation and induce differentiation.

Finally, the compounds of Formula (I), were characterized. Globally, the results show that the new compounds as well as combination of Alisertib and MP7 possess a comparable antiproliferative activity against GBM cell line. The same trend has been observed in stem cells. Moreover, further investigation on the ability to induce neurosphere differentiation has been performed. Data collected showed that the dual inhibition of PDK1 and AurA promotes CSC toward both a neuronal and a glial phenotype.

The invention will be now detailed by means of the following examples relating to the preparation of some invention compounds and to the evaluation of their activity against PDK1/AurA receptor.

### EXPERIMENTAL PARTS

### Example 1: Preparation of the Compounds of Formula (I)

Chemistry.

General material and methods. Commercial grade anhydrous solvents were used without further drying. Commercially available chemicals were purchased from Sigma-Aldrich or Alfa Aesar and used without further purification. Evaporation was performed in vacuum (rotating evaporator). Anhydrous Na₂SO₄ was always used as the drying agent. Flash chromatography was performed on Merck 60 Å high-purity grade silica gel (0.40-63 □m). Reactions were followed by TLC, performed on Merck aluminium silica gel (60 F254) sheets. Spots were viewed under a UV lamp (254 nm) or with the aid 10% phosphomolybdic acid in EtOH. Hydrogenation reactions were performed through HG2000 CLAIND® hydrogen generator. Celite® 545 was used as filter agent.

¹H, ¹³C and ¹⁹F NMR spectra were obtained using a Bruker Avance 400 spectrometer and were recorder at 400, 101 and 376 MHz, respectively. Chemical shifts are reported in parts per million (ppm) δ values, downfield from the internal reference tetramethylsilane (TMS) and referenced from solvent resonance as the internal standard: deuterochloroform [δ 7.26 (¹H spectra), δ 77.16 (¹³C spectra)]; deuterodimethylsulfoxide [δ 2.50 (¹H spectra), δ 39.52 (¹³C spectra)]; deuteromethanol [δ 3.31 (¹H spectra)]. Coupling constants J are reported in hertz (Hz). ¹⁹F and ¹³C NMR spectra are ¹H decoupled. ¹⁹F NMR spectra are unreferenced, corrected from Trifluoroacetic Acid (TFA) as external standard (−76.2 ppm). Signal patterns are indicated as follows: singlet (s), doublet (d), triplet (t), double-doublet (dd), double-triplet (dt), multiplet (m), broad singlet (br s), broad doublet (br d), broad triplet (br t) and broad multiplet (br m).

Abbreviation: DCM=dichloromethane TFA=Trifluoroacetic acid TBTU=N,N,N′,N′-Tetramethyl-O-(benzotriazol-1-yl)uronium tetrafluoroborate; DIPEA=N,N-Diisopropylethylamine; DMF=N,N-Dimethylformamide; rt=room temperature

Compounds IB35, DD21, DF8 were prepared according to

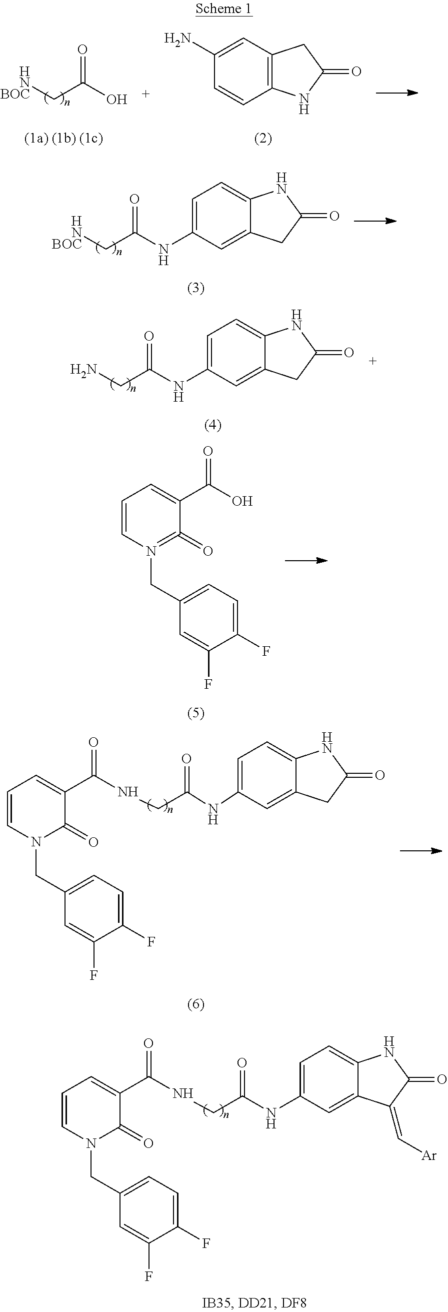

Wherein

n=1, Ar=imidazolyl IB35;

n=2 Ar=imidazolyl DD21;

n=3 Ar=imidazolyl DF8

### Preparation for n=1 of Intermediates (1a)(3a)(4a)(5)(6a) for the Preparation of IB35

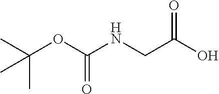

### 2-((tert-butoxycarbonyl)amino)acetic acid (1a)

To a solution of glycine (13.32 mmol) in 1M NaOH/iPrOH (4:3) was added Boc₂O (2.9 g, 13.32 mmol). The reaction, monitored by TLC, was stirred at room temperature for 2 h and then washed with Et₂O, acidified to pH 3.0 with 1N HCl and finally extracted several time with AcOEt. The organic layer was dried over anhydrous Na₂SO₄ and evaporated under reduced pressure. The crude product 4a was used for the next step without further purification (1.68 g, 9.59 mmol, 72% yield). ¹H-NMR (400 MHz, CDCl₃): δ 1.45 (s, 9H, Boc); 3.90-4.08 (m, 2H, CH₂); 5.07 (br s, 1H, NH) ppm. Anal. Calcd for C₇H₁₃NO₄: C, 47.99%; H, 7.48%; N, 8.00%; Found: C, 48.18%; H, 7.36%; N, 8.26%.

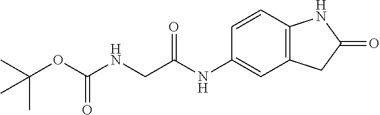 [SMILES: CC(C)(C)OC(=O)NCC(=O)Nc1ccc2c(c1)CC(=O)N2]

### tert-butyl (2-oxo-2-((2-oxoindolin-5-yl)amino)ethyl)carbamate (3a)

N-Boc derivative 1a (2.64 mmol) was reacted through a condensation reaction with 5-amino-indol-2-one (391 mg, 2.64 mmol), in the presence of the condensing agent TBTU (848 mg, 2.64 mmol) and DIPEA (5.28 mmol, 0.92 mL) as a base. The amide was obtained after purification of the crude product by column chromatography over silica gel using CHCl₃/MeOH 92:8 as the eluent (693 mg, 2.27 mmol, 86% yield). ¹H-NMR (400 MHz, DMSO-d₆): δ 1.39 (s, 9H, Boc); 3.46 (s, 2H, CH₂ indole); 3.68 (d, 2H, J=6.0 Hz, CH₂ glycine); 6.73 (d, 1H, J=8.2 Hz, Ar); 7.00 (t, 1H, J=6.0 Hz, NH); 7.32 (dd, 1H, J=2.0, 8.2 Hz, Ar); 7.49 (s, 1H, Ar); 9.74 (br s, 1H, NH); 10.27 (br s, 1H, NH) ppm. Anal. Calcd for C₁₅H₁₉N₃O₄: C, 59.01%; H, 6.27%; N, 13.76%; Found: C, 59.10%; H, 6.19%; N, 13.99%.

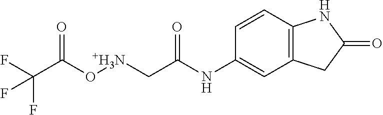 [SMILES: O=C(C[NH2+]OC(=O)C(F)(F)F)Nc1ccc2c(c1)CC(=O)N2]

### 2-oxo-2-((2-oxoindolin-5-yl)amino)ethanaminium 2,2,2-trifluoro-acetate(4a)

To a stirred suspension of 3a (2.27 mmol) in DCM (4.54 mL) cooled at −10° C./−20° C., was added TFA (4.54 mL). The reaction was monitored by TLC and reached completion in 3 h at the same temperature. Then the reaction mixture was evaporated to dryness to afford 4a as a trifluoroacetic salt, used for the following step without further purifications. ¹H-NMR (400 MHz, DMSO-d₆): δ 3.49 (s, 2H, CH₂ indole); 3.74 (d, 2H, J=5.6 Hz, CH₂ glycine); 6.78 (d, 1H, J=8.0 Hz, Ar); 7.33 (dd, 1H, J=2.2, 8.2 Hz, Ar); 7.47 (s, 1H, Ar); 8.09 (br s, 3H, NH₃⁺); 10.26 (br s, 1H, NH); 10.34 (br s, 1H, NH) ppm.

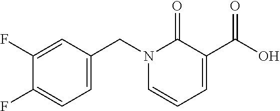 [SMILES: O=C(O)c1cccn(Cc2ccc(F)c(F)c2)c1=O]

### 1-(3,4-difluorobenzyl)-2-oxo-1,2-dihydropyridine-3-carboxylic acid (5)

A stirred suspension of NaH 60% dispersion in mineral oil (206 mg, 5.14 mmol), washed twice with distilled n-hexane and once with Et₂O under N₂ atmosphere, was treated dropwise with a solution containing the 2-hydroxynicotinic acid (600 mg, 4.31 mmol) in 5 mL of anhydrous DMF. The mixture was left under stirring at rt for 2 h and then 3,4-difluoro-benzylbromide (1.06 g, 5.14 mmol) was added and the mixture stirred and heated at 50° C. for 16 h. After, the mixture was concentrated under reduced pressure and the residue was treated with water to give a solid, which was collected by vacuum filtration. Next the solid was refluxed for 4 h in aq. 10% NaOH (10 mL) and the resulting mixture was cooled and made acid with 1N aq. HCl. The white solid formed was collected by filtration and washed with n-hexane and Et₂O, giving 5 as white solid (857 mg, 3.23 mmol, 75% yield). ¹H-NMR (400 MHz, DMSO-d₆): δ 5.30 (s, 2H, CH₂); 6.78 (t, 1H, J=6.9 Hz, Ar); 7.22-7.24 (m, 1H, Ar); 7.41-7.53 (m, 2H, Ar); 8.41 (d, 2H, J=6.9 Hz, Ar) ppm. Anal. Calcd for C₁₃H₉NO₃F₂: C, 58.87%; H, 3.42%; N, 5.28%; Found: C, 58.99%; H, 3.47%; N, 5.43%.

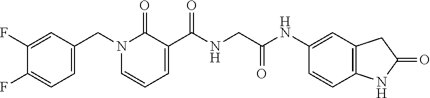 [SMILES: O=C(CNC(=O)c1cccn(Cc2ccc(F)c(F)c2)c1=O)Nc1ccc2c(c1)CC(=O)N2]

### Synthesis of 1-(3,4-difluorobenzyl)-2-oxo-N-(2-oxo-2-((2-oxoindolin-5-yl)amino)ethyl)-1,2-dihydropyridine-3-carboxamide (6a)

Starting from carboxylic acid 5 (154 mg, 0.58 mmol) and the 2-oxo-2-((2-oxoindolin-5-yl)amino)ethanaminium 2,2,2-trifluoro-acetate (0.58 mmol), the amide derivative was synthesized, using TBTU (187 mg, 0.58 mmol) as condensing agent. The solvents were evaporated under reduced pressure. The crude product was purified by column chromatography over silica gel using CHCl₃/MeOH 92:8 as the eluent to obtain the product as a white solid (63 mg, 0.14 mmol, 25% yield). mp: 257-262° C. ¹H-NMR (400 MHz, DMSO-d₆): δ 3.45 (s, 2H, CH₂ indole); 4.13 (d, 2H, J=5.2 Hz, CH₂ glycine); 5.23 (s, 2H, CH₂); 6.58 (t, 1H, J=6.9 Hz, Ar); 6.74 (d, 1H, J=8.4 Hz, Ar); 7.19-7.22 (m, 1H, Ar); 7.33 (dd, 1H, J=2.0, 8.4 Hz, Ar); 7.40-7.50 (m, 3H, Ar); 8.25 (dd, 1H, J=2.1, 6.9 Hz, Ar); 8.35 (dd, 1H, J=2.1, 6.9 Hz, Ar); 9.94 (br s, 1H, NH); 9.99 (t, 1H, J=5.2 Hz, NH); 10.28 (br s, 1H, NH) ppm. ¹³C-NMR (101 MHz, DMSO-d₆): δ 176.17, 166.69, 163.04, 161.09, 150.22, 147.83, 143.48, 143.19, 139.33, 134.18, 132.82, 126.05, 124.89, 120.20, 118.48, 117.65, 117.23, 116.44, 108.81, 106.45, 51.34, 43.05, 35.97 ppm. ¹⁹F-NMR (376 MHz; DMSO-d₆): δ −138.27 (d, 1F, J=24 Hz); −139.80 (d, 1F, J=24 Hz) ppm. Anal. Calcd for C₂₃H₁₈N₄O₄F₂: C, 61.06%; H, 4.01%; N, 12.38%; Found: C, 61.22%; H, 4.00%; N, 12.51%.

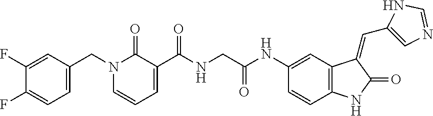 [SMILES: O=C(CNC(=O)c1cccn(Cc2ccc(F)c(F)c2)c1=O)Nc1ccc2c(c1)/C(=C/c1cnc[nH]1)C(=O)N2]

### (Z)—N-(2-((3-((1H-imidazol-5-yl)methylene)-2-oxoindolin-5-yl)amino)-2-oxoethyl)-1-(3,4-difluorobenzyl)-2-oxo-1,2-dihydro pyridine-3-carboxamide (IB35)

To a solution of 2-oxo-indole derivative (0.12 mmol) dissolved in iPrOH/DMF or absolute EtOH (5 mL), was added the appropriate carbaldehyde (0.12 mmol) and a catalytic amount of piperidine. The resulting solution was stirred and heated to reach 110° C. for 4 h, then the solution was evaporated to dryness. The residual material was purified by crystallization from iPrOH, affording the Z-isomer as a yellow solid (32 mg, 0.06 mmol, 52% yield). mp: 230-235° C. ¹H-NMR (400 MHz, DMSO-d₆): δ 4.17 (d, 2H, J=4.4 Hz, CH₂ glycine); 5.24 (s, 2H, CH₂); 6.60 (t, 1H, J=6.9 Hz, Ar); 6.85 (d, 1H, J=8.8 Hz, Ar); 7.19-7.21 (m, 2H, Ar); 7.43-7.50 (m, 3H, Ar); 7.71 (s, 1H, Ar); 7.75 (s, 1H, Ar); 8.01 (s, 1H, Ar); 8.05 (s, 1H, Ar); 8.27 (dd, 1H, J=2.0, 6.9 Hz, Ar); 8.37 (d, 1H, J=2.0, 6.9 Hz, Ar); 10.61 (br s, 2H, NH); 10.95 (br s, 1H, NH) ppm. ¹³C-NMR (101 MHz, DMSO-d₆): δ 169.05, 166.93, 163.12, 161.16, 150.41, 147.90, 143.57, 143.31, 139.62, 138.85, 135.79, 134.32, 133.20, 128.07, 124.93, 124.47, 122.90, 120.25, 120.10, 119.67, 117.73, 117.23, 111.17, 109.86, 106.52, 51.45, 43.14 ppm. ¹⁹F-NMR (376 MHz; DMSO-d₆): δ −138.25 (d, 1F, J=24 Hz); −139.80 (d, 1F, J=24 Hz) ppm. Anal. Calcd for C₂₇H₂₇N₆O₄F₂: C, 61.13%; H, 3.80%; N, 15.84%; Found: C, 61.15%; H, 4.03%; N, 15.89%.

### Preparation of Intermediates for n=2 of Intermediates (1b)(3b)(4b)(6b) for DD21

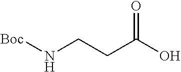 [SMILES: CC(C)(C)OC(=O)NCCC(=O)O]

### 3-((tert-butoxycarbonyl)amino)propanoic acid (1b)

To a solution of β-alanine (1.00 g, 11.24 mmol) in NaOH 1M/IPrOH 4:3 (14 mL) at 0° C. was added Boc₂O (2.45 g, 11.24 mmol). The reaction, monitored by TLC, was stirred at rt for 2 h and then washed with Et₂O, acidified to pH 3.0 with 1N HCl and finally extracted several time with AcOEt. The organic layer was dried over anhydrous Na₂SO₄ and evaporated under reduced pressure. The crude product (1.03 g, 5.46 mmol, 50% yield) was used for the next step without further purification. ¹H-NMR: (CDCl₃): δ 1.43 (s, 9H, Boc); 2.52-2.59 (m, 2H, CH₂); 3.39-3.41 (m, 2H, CH₂); 5.05 (br s, 1H, NH) ppm. Anal. Calcd for C₈H₁₅NO₄: C, 50.78%; H, 7.99%; N, 7.40%; Found: C, 51.02%; H, 8.02%; N, 7.54%.

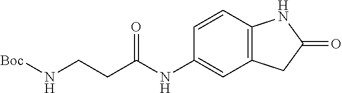 [SMILES: CC(C)(C)OC(=O)NCCC(=O)Nc1ccc2c(c1)CC(=O)N2]

### tert-butyl (3-oxo-3-((2-oxoindolin-5-yl)amino)propyl)carbamate (3b)

To a solution of N-Boc amine 1b (578.34 mg, 3.06 mmol), in dry DMF (5 mL), under N₂ atmosphere and cooled to 0° C., were added TBTU (982.54 mg, 3.06 mmol) and DIPEA (1.07 mL, 6.12 mmol). After 30′ a 0° C., the amine salt 2b (0.58 mmol) was added and the temperature was kept at 0° C. for additional 30′. Later the mixture was slowly warmed to rt and left under stirring at rt overnight. Once TLC verified the disappearance of the precursor, the organic solvent was evaporated under vacuum and the crude product was purified by flash chromatography over silica gel, using CHCl₃/MeOH (95:5) as the eluent, to obtain pure 3b as a white solid (806 mg, 2.53 mmol, 83% yield). ¹H-NMR (DMSO-d₆): δ 1.39 (s, 9H, Boc); 2.42 (t, 2H, J=7.2 Hz, CH₂); 3.16-3.22 (m, 2H, CH₂); 3.45 (s, 2H, CH₂); 6.71 (d, 1H, J=8.4 Hz, Ar); 6.84-6.88 (br t, 1H, J=5.6 Hz, NH); 7.32 (dd, 1H, J=1.6, 8.4 Hz); 7.50 (s, 1H, Ar); 9.78 (br s, 1H, NH); 10.28 (br s, 1H, NH) ppm. Anal. Calcd for C₁₆H₂₁N₃O₄: C, 60.37%; H, 6.33%; N, 13.20%; Found: C, 60.17%; H, 6.23%; N, 13.22%.

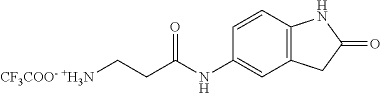

### 3-oxo-3-((2-oxoindolin-5-yl)amino)propan-1-aminium 2,2,2-trifluoroacetate (4b)

To a stirred suspension of 3b (796 mg, 2.50 mmol) in DCM (5 mL) cooled at −10° C./−20° C., was added TFA (5 mL). The reaction was monitored by TLC and reached completion in 4 h at the same temperature. Then the reaction mixture was evaporated to dryness to afford 4b as a trifluoroacetic salt, used for the following step without further purifications. ¹H-NMR (DMSO-d₆): δ 2.67 (t, 2H, J=6.8 Hz, CH₂); 3.06-3.11 (m, 2H, CH₂); 3.49 (s, 2H, CH₂); 6.75 (d, 1H, J=8.4 Hz, Ar); 7.33 (dd, 1H, J=2.0, 8.4 Hz, Ar); 7.50 (s, 1H, Ar); 7.79 (br s, 3H, NH₃⁺); 10.02 (br s, 1H, NH); 10.33 (br s, 1H, NH) ppm.

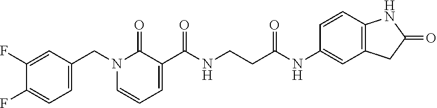 [SMILES: O=C(CCNC(=O)c1cccn(Cc2ccc(F)c(F)c2)c1=O)Nc1ccc2c(c1)CC(=O)N2]

### 1-(3,4-difluorobenzyl)-2-oxo-N-(3-oxo-3-((2-oxoindolin-5-yl)amino)propyl)-1,2-dihydropyridine-3-carboxamide (6b)

To a solution of carboxylic acid 5 (500 mg, 1.89 mmol), in dry DMF (5 mL), under N₂ atmosphere and cooled to 0° C., were added TBTU (607 mg, 1.89 mmol) and DIPEA (5 mL). After 30′ a 0° C., the amine salt 4b (629 mg, 1.89 mmol) was added and the temperature was kept at 0° C. for additional 30′. Later the mixture was slowly warmed to room temperature and left under stirring at rt overnight. Once TLC verified the disappearance of the precursor, the organic solvent was evaporated under vacuum and the crude product was purified by flash chromatography over silica gel, using CHCl₃/MeOH (92:8) as the eluent, to obtain pure 6b as a white solid (364 mg, 0.81 mmol 43% yield). ¹H-NMR (DMSO-d₆): δ 2.52-2.56 (m, 2H, CH₂); 3.44 (s, 2H, CH₂); 3.54-3.57 (m, 2H, CH₂); 5.19 (s, 2H, CH₂); 6.56 (t, 1H, J=6.9 Hz, Ar); 6.71 (d, 1H, J=8.4 Hz, Ar); 7.14-7.17 (m, 1H, Ar); 7.30 (d, 1H, J=8.4 Hz, Ar); 7.35-7.45 (m, 2H, Ar); 7.50 (s, 1H, Ar); 8.20 (d, 1H, J=6.9 Hz, Ar); 8.35 (d, 1H, J=6.9 Hz, Ar); 9.77-9.79 (br m, 1H, NH); 9.81 (br s, 1H, NH); 10.28 (br s, 1H, NH) ppm. ¹³C-NMR (DMSO-d₆): δ 176.28, 169.02, 162.97, 161.18, 150.33, 147.88, 143.47, 143.02, 139.25, 134.22, 133.20, 125.99, 124.85, 120.38, 118.61, 117.69, 117.20, 116.63, 108.81, 106.59, 51.25, 36.12, 36.03, 35.15 ppm. Anal. Calcd for C₂₄H₂₀N₄O₄F₂: C, 61.80%; H, 4.32%; N, 12.01%; Found: C, 62.03%; H, 4.45%; N, 12.09%.

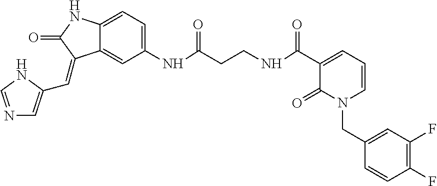 [SMILES: O=C(CCNC(=O)c1cccn(Cc2ccc(F)c(F)c2)c1=O)Nc1ccc2c(c1)/C(=C/c1cnc[nH]1)C(=O)N2]

### (Z)—N-(3-((3-((1H-imidazol-5-yl)methylene)-2-oxoindolin-5-yl)amino)-3-oxopropyl)-1-(3,4-difluorobenzyl)-2-oxo-1,2-dihydropyridine-3-carboxamide (DD21)

To a solution of 2-oxo-indole derivative 6b (50 mg, 0.11 mmol) dissolved in iPrOH/DMF, was added the 4-imidazolecarbaldehyde (11 mg, 0.11 mmol) and a catalytic amount of piperidine. The resulting solution was stirred and heated to reach 110° C. for 4 h, then the solution was evaporated to dryness. The residual material was purified by crystallization from iPrOH, affording the Z-isomer as an orange solid (29 mg, 0.05 mmol 49% yield). Mp: 295-300° C. ¹H-NMR (DMSO-d₆): δ 2.58-2.60 (m, 2H, CH₂); 3.57-3.61 (m, 2H, CH₂); 5.19 (s, 2H, CH₂); 6.57 (t, 1H, J=6.8 Hz, Ar); 6.82 (d, 1H, J=8.0 Hz, Ar); 7.14-7.21 (m, 2H, Ar); 7.33-7.45 (m, 2H, Ar); 7.71-7.73 (m, 2H, Ar); 8.00-8.02 (m, 2H, Ar); 8.19 (d, 1H, J=6.8 Hz, Ar); 8.36 (d, 1H, J=6.8 Hz, Ar); 9.79-9.81 (br m, 1H, NH); 9.90 (br s, 1H, NH); 10.93 (br s, 1H, NH) ppm. ¹³C-NMR (DMSO-d₆): δ 169.18, 169.02, 162.96, 161.17, 150.29, 147.87, 143.45, 143.00, 138.75, 137.90, 135.69, 134.17, 133.48, 132.37, 124.85, 122.74, 120.37, 120.15, 119.82, 117.64, 117.15, 111.26, 109.77, 106.57, 51.22, 36.08, 35.15 ppm. Anal. Calcd for C₂₈H₂₈N₆O₄F₂: C, 61.76%; H, 4.07%; N, 15.43%; Found: C, 61.88%; H, 4.15%; N, 15.59%.

### Preparation of Intermediate for n=3 of Intermediates (1c)(3c)(4c)(6c) for DF8

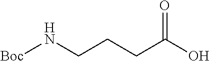 [SMILES: CC(C)(C)OC(=O)NCCCC(=O)O]

### 4-((tert-butoxycarbonyl)amino)butanoic acid (1c)

To a solution of γ-amminobutirric acid (1.00 g, 9.70 mmol) in NaOH 1M/IPrOH 4:3 (14 mL) at 0° C. was added Boc₂O (2.12 g, 9.70 mmol). The procedure followed is the same described for derivative 1a. The crude product (921 mg, 4.53 mmol, 50% yield) was used for the next step without further purification. ¹H NMR (CDCl₃): δ 1.44 (s, 9H, CH₃); 1.79-1.86 (m, 2H, CH₂); 2.40 (t, 2H, J=7.2 Hz, CH₂); 3.20-3.18 (m, 2H, CH₂) ppm. Anal. Calcd for C₉H₁₇NO₄: C, 53.19%; H, 8.43%; N, 6.89%; Found: C, 53.07%; H, 8.57%; N, 6.93%.

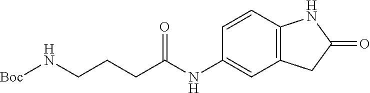 [SMILES: CC(C)(C)OC(=O)NCCCC(=O)Nc1ccc2c(c1)CC(=O)N2]

### tert-butyl (4-oxo-4-((2-oxoindolin-5-yl)amino)butyl)carbamate (3c)

N-Boc derivative 1c (413 mg, 2.03 mmol) was reacted through a condensation reaction with 5-amino-indol-2-one (300 mg, 2.03 mmol), in the presence of TBTU (652 mg, 2.03 mmol). The procedure followed is the same as described for derivative 3c. Final compound (726 mg, 1.92 mmol, 95% yield) has been purified by column chromatography over silica gel using CHCl₃/MeOH (95:5) as the eluent.

¹H NMR (DMSO-d₆): δ 1.37 (s, 9H, CH₃); 1.65-1.69 (m, 2H, CH₂); 2.24 (t, 2H, J=7.6 Hz, CH₂); 2.92-2.97 (m, 2H, CH₂); 3.44 (s, 2H, CH₂); 6.71 (d, 1H, J=8.2 Hz, Ar); 6.79 (br s, 1H, NH); 7.30 (d, 1H, J=8.2 Hz, Ar); 7.49 (s, 1H, Ar); 9.68 (br s, 1H, NH); 10.23 (br s, 1H, NH) ppm. Anal. Calcd for C₁₇H₂₃N₃O₄: C, 61.25%; H, 6.95%; N, 12.60%; Found: C, 60.98%; H, 6.89%; N, 12.41%.

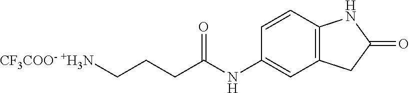

### 4-oxo-4-((2-oxoindolin-5-yl)amino)butan-1-aminium 2,2,2-trifluoroacetate (4c)

To a stirred suspension of 3c (604.3 mg, 1.81 mmol) in DCM (4.54 mL) cooled at −10° C./−20° C., was added TFA (3.63 mL). The reaction was carried out as described for compound 3b. Derivative 4c was obtained as a trifluoroacetic salt, used for the following step without further purifications.

¹H NMR (DMSO-d₆): δ 1.82-1.85 (m, 2H, CH₂); 2.38 (t, 2H, J=6.8 Hz, CH₂); 2.83-2.85 (m, 2H, CH₂); 3.45 (s, 2H, CH₂); 6.73 (d, 1H, J=8.0 Hz, Ar); 7.31 (d, 1H, J=8.0 Hz, Ar), 7.50 (s, 1H, Ar); 7.76 (br s, 3H, NH₃⁺); 9.84 (br s, 1H, NH); 10.29 (br s, 1H, NH) ppm.

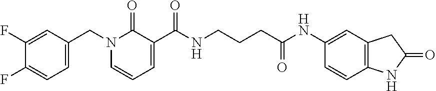 [SMILES: O=C(CCCNC(=O)c1cccn(Cc2ccc(F)c(F)c2)c1=O)Nc1ccc2c(c1)CC(=O)N2]

### 1-(3,4-difluorobenzyl)-2-oxo-N-(4-oxo-4-((2-oxoindolin-5-yl)amino)butyl)-1,2-dihydropyridine-3-carboxamide. (6c)

Carboxylic acid 5 (480 mg, 1.81 mmoli) was reacted through a condensation reaction with the amine salt 4c (480 mg, 1.81 mmol), in the presence of TBTU (581.16 mg, 1.81 mmoli). The procedure followed is the same as described for derivative 6b. The crude product was purified by flash chromatography over silica gel, using CHCl₃/MeOH (92:8) as the eluent, to obtain pure 6c as a white solid (680 mg, 1.42 mmol, 79% yield).

Mp: 199-204° C. ¹H NMR (DMSO-d₆): δ 1.76-1.84 (m, 2H, CH₂); 2.30 (t, 2H, J=7.6 Hz, CH₂); 3.34-3.36 (m, 2H, CH₂); 3.43 (s, 2H, CH₂); 5.20 (s, 2H, CH₂); 6.57 (t, 1H, J=7.2 Hz, Ar); 6.70 (d, 1H, J=8.4 Hz, Ar); 7.16-7.19 (m, 1H, Ar); 7.30 (dd, 1H, J=2.0, 8.4 Hz, Ar); 7.38-7.48 (m, 3H, Ar); 8.21 (dd, 1H, J=2.2, 7.2 Hz, Ar); 8.34 (dd, 1H, J=2.2, 7.2 Hz, Ar); 9.66 (br t, 1H, J=5.8 NH); 9.74 (br s, 1H, NH); 10.25 (br s, 1H, NH) ppm. ¹³C-NMR (DMSO-d₆): δ 176.23; 170.15; 162.98; 161.23; 150.31; 147.87; 143.40; 142.92; 139.06; 134.21; 133.38; 125.90; 124.85; 120.49; 118.42; 117.70; 117.20; 116.46; 108.73; 106.60; 51.26; 38.29; 36.01; 33.85; 25.41 ppm. ¹⁹F-NMR (DMSO-d₆): δ −138.23 (d, 1F, J=22 Hz); −139.82 (d, 1F, J=24 Hz) ppm. Anal. Calcd for C₂₅H₂₂N₄O₄F₂: C, 62.50%; H, 4.62%; N, 11.66%; Found: C, 62.32%; H, 4.79%; N, 11.81%.

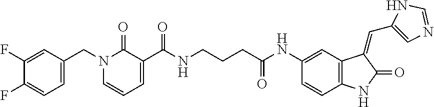 [SMILES: O=C(CCCNC(=O)c1cccn(Cc2ccc(F)c(F)c2)c1=O)Nc1ccc2c(c1)/C(=C/c1cnc[nH]1)C(=O)N2]

### (Z)—N-(4-((3-((1H-imidazol-5-yl)methylene)-2-oxoindolin-5-yl)amino)-4-oxobutyl)-1-(3,4-difluorobenzyl)-2-oxo-1,2-dihydropyridine-3-carboxamide (DF8)

To a solution of 2-oxo-indole derivative 6c (70 mg, 0.15 mmol) dissolved in iPrOH/DMF, was added the 4-imidazole-carbaldehyde (14 mg, 0.15 mmol) and a catalytic amount of piperidine. The procedure followed is the same described for compound IB35. The residual material was purified by crystallization from iPrOH and subsequent trituration in (iPr)₂O, affording the final product DF8 (55 mg, 0.10 mmol, 66% yield) affording the orange solid as the Zisomer. Mp: 240-244° C. ¹H NMR (DMSO-d₆): δ: 1.80-1.85 (m, 2H, CH₂); 2.34 (t, 2H, J=7.2 Hz, CH₂); 3.35-3.39 (m, 2H, CH₂); 5.19 (s, 2H, CH₂); 6.57 (t, 1H, J=7.0 Hz, Ar); 6.80 (d, 1H, J=8.4 Hz, Ar); 7.18-7.21 (m, 2H, Ar); 7.38-7.46 (m, 2H, Ar); 7.65-7.75 (m, 2H, Ar); 7.95-8.05 (m, 2H, Ar); 8.20 (dd, 1H, J=1.8, 7.0 Hz, Ar); 8.35 (dd, 1H, J=1.8, 7.0 Hz, Ar); 9.68 (br t, 1H, J=5.6 Hz, NH); 9.84 (br s, 1H, NH); 10.89 (br s, 1H, NH) ppm. ¹³C-NMR (DMSO-d₆): δ 170.33; 169.04; 162.99; 161.24; 150.05; 147.87; 143.41; 142.91; 139.52; 138.73; 134.20; 133.68; 124.85; 124.30; 122.70; 120.50; 120.20; 119.77; 117.70; 117.20; 111.22; 109.70; 106.60; 51.27; 38.31; 33.79; 25.38 ppm. ¹⁹F-NMR (DMSO-d₆): δ: −138.22 (d, 1F, J=24 Hz); −139.81 (d, 1F, J=24 Hz) ppm. Anal. Calcd for C₂₉H₂₉N₆O₄F₂: C, 62.36%; H, 4.33%; N, 15.05%; Found: C, 62.66%; H, 4.21%; N, 15.39%.

Compound SA16 was prepared as reported in Scheme 2

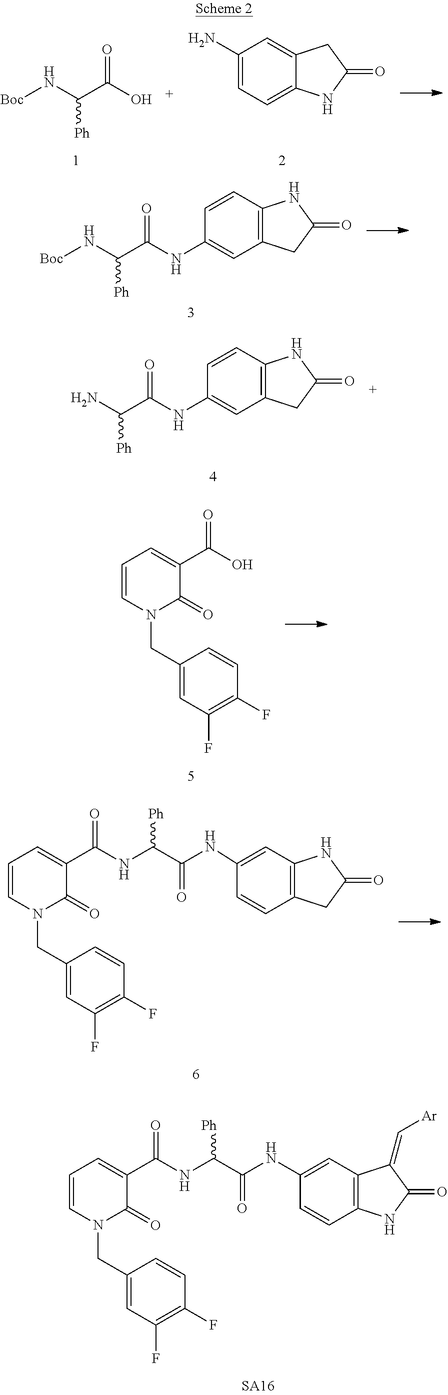

Wherein Ar is imidazolyl

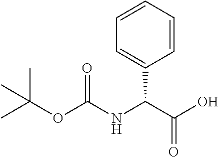 [SMILES: CC(C)(C)OC(=O)N[C@@H](C(=O)O)c1ccccc1]

### (R)-2-((tert-butoxycarbonyl)amino)-2-phenylacetic acid (1)

Compound 1 was synthesized from (R)-(−)-2-phenylglycine (2 g, 13.32 mmol) and Boc₂O (2.9 g, 13.32 mmol) in a solution 1M NaOH/iPrOH (4:3) following the same procedure described above for the preparation of 1a. 1H-NMR (400 MHz, CDCl₃): δ 1.21 (s, 6H, Boc); 1.43 (s, 3H, Boc); 5.12-5.51 (m, 1H); 7.29-7.44 (m, 5H); 7.96 (br s, 1H, NH) ppm. Anal. Calcd for C₁₃H₁₇NO₄: C, 62.14%; H, 6.82%; N, 5.57%; Found: C, 62.18%; H, 7.06%; N, 5.86%.

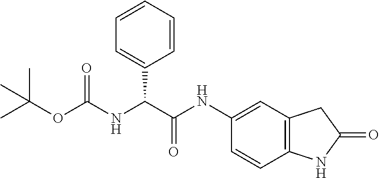 [SMILES: CC(C)(C)OC(=O)N[C@@H](C(=O)Nc1ccc2c(c1)CC(=O)N2)c1ccccc1]

### (R)-tert-butyl (2-oxo-2-((2-oxoindolin-5-yl)amino)-1-phenylethyl)-carbamate (3)

N-Boc derivative 1 (663 mg, 2.64 mmol) was reacted through a condensation reaction with 5-amino-indol-2-one (391 mg, 2.64 mmol), in the presence of TBTU (848 mg, 2.64 mmol). The procedure followed is the same as described for derivative 3a. Final compound has been purified by column chromatography over silica gel using CHCl3/MeOH (92:8) as the eluent. 1H-NMR (400 MHz, DMSO-d₆): δ 1.39 (s, 9H, Boc); 3.44 (s, 2H, CH₂ indole); 5.30-5.33 (m, 1H); 6.72 (d, 1H, J=8.4 Hz, Ar); 7.26-7.36 (m, 4H, Ar); 7.44-7.48 (m, 4H, Ar); 10.09 (br s, 1H, NH); 10.29 (br s, 1H, NH) ppm. Anal. Calcd for C21H23N3O4: C, 66.13%; H, 6.08%; N, 11.02%; Found: C, 66.17%; H, 6.23%; N, 11.21%.

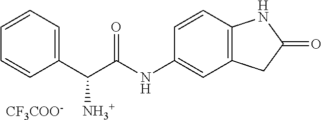

### (R)-2-amino-N-(2-oxoindolin-5-yl)-2-phenylacetamide (4)

To a stirred suspension of 3 (866 mg, 2.27 mmol) in DCM (4.54 mL) cooled at −10° C./−20° C., was added TFA (4.54 mL). Derivative 4 was obtained as a trifluoroacetic salt, used for the following step without further purifications. 1H-NMR (400 MHz, DMSO-d₆): δ 3.47 (s, 2H, CH₂); 5.03-5.05 (m, 1H); 6.76 (d, 1H, J=8.4 Hz, Ar); 7.29 (dd, 1H, J=2.0, 8.4 Hz, Ar); 7.42-7.50 (m, 3H, Ar); 7.57-7.59 (m, 2H, Ar); 8.72-8.74 (m, 1H, Ar); 10.36 (br s, 1H, NH); 10.47 (br s, 1H, NH) ppm.

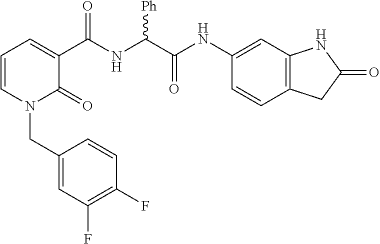 [SMILES: O=C1Cc2ccc(NC(=O)C(NC(=O)c3cccn(Cc4ccc(F)c(F)c4)c3=O)c3ccccc3)cc2N1]

### Synthesis of (R)-1-(3,4-difluorobenzyl)-2-oxo-N-(2-oxo-2-((2-oxoindolin-5-yl)amino)-1-phenylethyl)-1,2-dihydropyridine-3-carboxamide (6)

The amide 6 was synthesized starting from carboxylic acid 5 (154 mg, 0.58 mmol) and the amine salt 4 (0.58 mmol), using TBTU (187 mg, 0.58 mmol) as condensing reagent. The crude product was purified by column chromatography over silica gel using CHCl3/MeOH (92:8) as the eluent to obtain the final product as a white solid (201 mg, 0.38 mmol, 66% yield). Mp: 160-165° C. 1H NMR (400 MHz, DMSO-d₆): δ 3.44 (s, 2H, CH₂ indole); 5.23-5.30 (m, 2H, CH₂); 5.77 (d, 1H, J=7.4 Hz); 6.59 (t, 1H, J=7.0 Hz, Ar); 6.72 (d, 1H, J=8.4 Hz, Ar); 7.19-7.22 (m, 1H, Ar); 7.28-7.49 (m, 9H, Ar); 8.25 (dd, 1H, J=2.0, 6.8 Hz, Ar); 8.35 (dd, 1H, J=2.0, 6.8 Hz, Ar); 10.31 (br s, 1H, NH); 10.34 (br s, 1H, NH); 10.65 (d, 1H, J=7.4 Hz, NH) ppm. 13C NMR (101 MHz, DMSO-d₆): δ 176.25, 167.78, 162.32, 161.29, 150.36, 147.91, 143.80, 143.52, 139.70, 138.69, 134.21, 132.60, 128.68, 127.86, 126.78, 126.22, 124.87, 119.91, 118.66, 117.77, 117.19, 116.55, 108.89, 106.68, 57.07, 51.32, 36.00 ppm. 19F-NMR (376 MHz; DMSO-d₆): δ 138.15 (d, 1F, J=24 Hz); 139.78 (d, 1F, J=24 Hz) ppm. Anal. Calcd for C₂₉H₂₂N₄O₄F₂: C, 65.90%; H, 4.20%; N, 10.60%; Found: C, 66.10%; H, 4.38%; N, 10.91%.

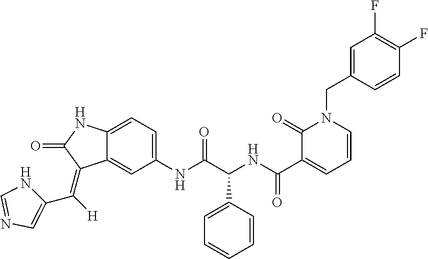 [SMILES: O=C1Nc2ccc(NC(=O)[C@H](NC(=O)c3cccn(Cc4ccc(F)c(F)c4)c3=O)c3ccccc3)cc2/C1=C/c1cnc[nH]1]

### (Z)—(R)—N-(2-((3-((1H-imidazol-5-yl)methylene)-2-oxoindolin-5-yl)amino)-2-oxo-1-phenylethyl)-1-(3,4-difluorobenzyl)-2-oxo-1,2-dihydropyridine-3-carboxamide (SA16)

To a solution of 2-oxo-indole derivative 6 (63 mg, 0.12 mmol) dissolved in absolute EtOH, was added the 4-imidazolecarbaldehyde (12 mg, 0.12 mmol) and a catalytic amount of piperidine. The procedure followed is the same described for derivative IB35. The residual material was purified by crystallization from EtOH, affording the Z-isomer as a yellow solid (41 mg, 0.07 mmol, 57% yield). Mp: 248-255° C. 1H-NMR (400 MHz, DMSO-d₆): δ 5.20-5.31 (m, 2H, CH₂); 5.81 (d, 1H, J=7.2 Hz, CH□); 6.60 (t, 1H, J=6.8 Hz, Ar); 6.83 (d, 1H, J=8.0 Hz, Ar); 7.19-7.21 (m, 2H, Ar); 7.29-7.52 (m, 8H, Ar); 7.71 (s, 1H, Ar); 7.78 (s, 1H, Ar); 8.01 (s, 1H, Ar); 8.04 (s, 1H, Ar); 8.27 (dd, 1H, J=2.0, 6.8 Hz, Ar); 8.35 (dd, 1H, J=2.0, 6.8 Hz, Ar); 10.46 (br s, 1H, NH); 10.72 (d, 1H, J=7.2 Hz, NH); 10.97 (br s, 1H, NH) ppm. 13C-NMR (101 MHz, DMSO-d₆): δ 169.03, 167.95, 162.32, 161.30, 150.35, 147.91, 143.82, 143.57, 139.68, 138.95, 138.72, 136.04, 134.24, 132.92, 128.70, 128.07, 127.89, 126.83, 124.87, 124.51, 123.16, 119.90, 119.77, 117.79, 117.15, 111.16, 109.88, 106.68, 57.06, 51.36 ppm. 19F-NMR (376 MHz; DMSO-d₆): δ −138.15 (d, 1F, J=24 Hz); −139.76 (d, 1F, J=24 Hz) ppm. Anal. Calcd for C₃₃H₃₃N₆O₄F₂: C, 65.34%; H, 3.99%; N, 13.85%; Found: C, 65.25%; H, 4.03%; N, 13.79%.

Compounds SST200, VI8, VI23, VI18 were prepared according to

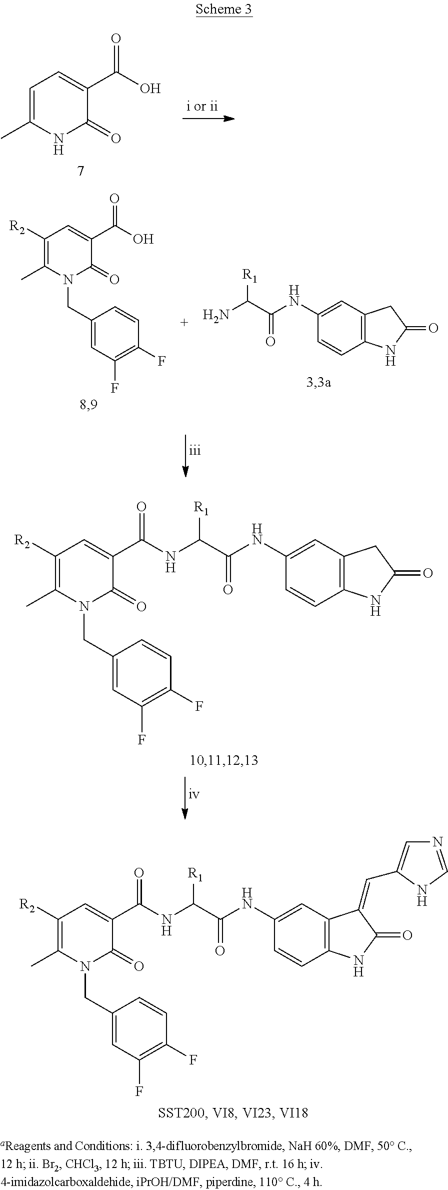

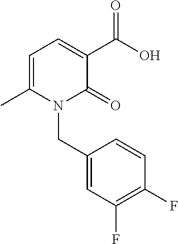 [SMILES: Cc1ccc(C(=O)O)c(=O)n1Cc1ccc(F)c(F)c1]

### 1-(3,4-difluorobenzyl)-6-methyl-2-oxo-1,2-dihydropyridine-3-carboxylic acid (8)

To a solution of 2-hydroxy-nicotinic acid 7 (6.53 mmol, 1 g) in DMF anhydrous (5 ml) under N₂ current, CsF (19.59 mmol, 2975.3 mg) was added. The reaction mixture was allowed to stir at r.t. for 1 h. After this period, 3,4-difluoro-benzyl bromide (7.83 mmol, 1 ml) was added and the resulting mixture was heated at 50° C. for 16 h. The solvent was evaporated under reduced pressure and the residue was triturated in H2O and filtered on a pad. This solid was then refluxed for 4 h in a solution of NaOH 10% (10 mL). The cooled solution was acidified with 1N HCl and the precipitate was collected by filtration and then crushed with Et₂O to provide the pure 8 derivative. C₁₄H₁₁F₂NO₃ (Yield: 40%)¹H-NMR (DMSO): 2.45 (s, 3H, CH₃); 5.42 (s, 2H, CH₂); 6.71 (d, 1H, J=7.6 Hz, Ar); 7.00-7.03 (m, 1H, Ar); 7.32-7.44 (m, 2H, Ar); 8.33 (d, 1H, J=7.6 Hz, Ar) ppm

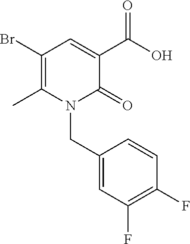 [SMILES: Cc1c(Br)cc(C(=O)O)c(=O)n1Cc1ccc(F)c(F)c1]

### 5-bromo-1-(3,4-difluorobenzyl)-6-methyl-2-oxo-1,2-dihydropyridine-3-carboxylic acid (9)

To a solution of 5-nitro-2-oxindole (2.607 mmol, 728 mg) in CHCl3, 0.40 mL of Br2 was added. The reaction is allowed to react for 16 h at rt. Then, a saturated sodium sulfate solution was added. The organic phase was evaporated and the solid triturated in hexane to yield the pure product. C₁₄H₁₀BrF₂NO₃ (Yield: 84.2%)¹H-NMR (DMSO): δ 2.56 (s, 3H, CH₃); 5.50 (s, 2H, CH₂); 7.07-7.09 (m, 1H, Ar); 7.36-7.45 (m, 2H, Ar); 8.41 (s, 1H, Ar); ppm.

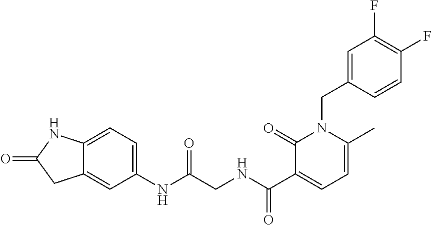 [SMILES: Cc1ccc(C(=O)NCC(=O)Nc2ccc3c(c2)CC(=O)N3)c(=O)n1Cc1ccc(F)c(F)c1]

### 1-(3,4-difluorobenzyl)-6-methyl-2-oxo-N-(2-oxo-2-((2-oxoindolin-5-yl)amino)ethyl)-1,2-dihydropyridine-3-carboxamide (10)

Compound 10 was synthesised following the same procedure described in scheme 1. The crude product 10 was purified by precipitation from H₂O/E₂O. C₂₄H₂₀F₂N₄O₄ (Yield: 99.7%). ¹H-NMR (DMSO): δ 2.42 (s, 3H, CH₃); 3.46 (s, 2H, CH₂ indole); 4.14 (d, 2H, J=4.4 Hz, CH₂ glycine); 5.39 (s, 2H, CH₂); 6.52 (d, 1H, J=7.4 Hz, Ar); 6.74 (d, 1H, J=8.4 Hz, Ar); 6.91-7.05 (m, 1H, Ar); 7.29-7.49 (m, 4H, Ar); 8.29 (d, 1H, J=7.4 Hz, Ar); 9.95 (s, 1H, NH); 9.99-10.05 (m, 1H, NH); 10.28 (br s, 1H, NH) ppm. ¹³C-NMR (DMSO): δ 176.3; 166.9; 163.4; 162.3; 152.5; 142.8; 139.4; 133.9; 132.9; 126.1; 123.1; 118.6; 117.9; 117.8; 117.2; 116.5; 115.9; 115.8; 108.9; 107.7; 46.4; 43.1; 36.0; 20.7 ppm.

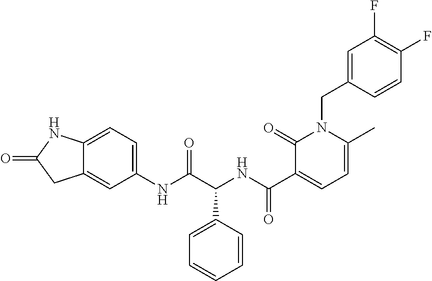 [SMILES: Cc1ccc(C(=O)N[C@@H](C(=O)Nc2ccc3c(c2)CC(=O)N3)c2ccccc2)c(=O)n1Cc1ccc(F)c(F)c1]

### (R)-1-(3,4-difluorobenzyl)-6-methyl-2-oxo-N-(2-oxo-2-((2-oxoindolin-5-yl)amino)-1-phenylethyl)-1,2-dihydropyridine-3-carboxamide (11)

Compound 11 was synthesised following the same procedure described for compound 10. C₃₀H₂₄F₂N₄O₄ (yield: 98.1%)¹H-NMR (DMSO): δ 2.41 (s, 3H, CH₃); 3.43 (s, 2H, CH₂ indole); 5.35-5.46 (m, 2H, CH₂); 5.78 (d, 1H, J=7.2 Hz, CH); 6.52 (d, 1H, J=7.6 Hz, Ar); 6.72 (d, 1H, J=8.0 Hz, Ar); 6.91-7.03 (m, 1H, Ar); 7.30-7.50 (m, 9H, Ar); 8.28 (d, 1H, J=7.6 Hz, Ar); 10.29-10.34 (m, 2H, NH); 10.64 (br d, 1H, J=7.2 Hz; NH) ppm.

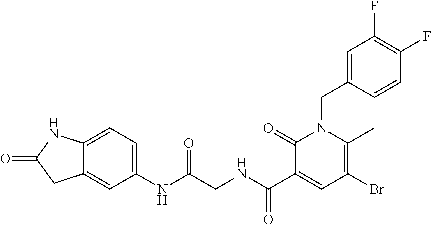 [SMILES: Cc1c(Br)cc(C(=O)NCC(=O)Nc2ccc3c(c2)CC(=O)N3)c(=O)n1Cc1ccc(F)c(F)c1]

### 5-bromo-1-(3,4-difluorobenzyl)-6-methyl-2-oxo-N-(2-oxo-2-((2-oxoindolin-5-yl)amino)-ethyl)-1,2-dihydropyridine-3-carboxamide (12)

The final product was synthesised following the same procedure described for derivative 11. The crude 12 was purified by column chromatography, eluting with a mixture of CHCl3/MeOH 95/5. C₂₄H₁₉BrF₂N₄O₄ (Yield: 93.44%)¹H-NMR (DMSO): δ 2.53 (s, 3H, CH₃); 3.45 (s, 2H, CH₂ indole); 4.15 (d, 2H, J=5.2 Hz, CH₂ glycine); 5.46 (s, 2H, CH₂); 6.73 (d, 1H, J=8.0 Hz, Ar); 6.96-7.05 (m, 1H, Ar); 7.31-7.45 (m, 3H, Ar); 7.47 (s, 1H, Ar); 8.38 (s, 1H, Ar); 9.91 (br t, 1H, J=5.2 Hz, NH); 9.99 (br s, 1H, NH); 10.31 (br s, 1H, NH) ppm.

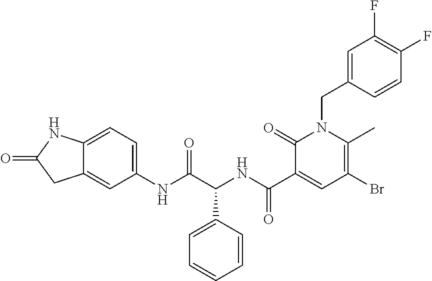 [SMILES: Cc1c(Br)cc(C(=O)N[C@@H](C(=O)Nc2ccc3c(c2)CC(=O)N3)c2ccccc2)c(=O)n1Cc1ccc(F)c(F)c1]

### (R)-5-bromo-1-(3,4-difluorobenzyl)-6-methyl-2-oxo-N-(2-oxo-2-((2-oxoindolin-5-yl)amino)-1-phenylethyl)-1,2-dihydropyridine-3-carboxamide (13)

The final product was synthesised following the same procedure described for derivative 12 C₃₀H₂₃BrF₂N₄O₄ (yield: 73.9%). ¹H-NMR (DMSO): δ 2.41 (s, 3H, CH₃); 3.44 (s, 2H, CH₂ indole); 5.42-5.51 (m, 2H, CH₂); 5.78-5.82 (m, 1H, CH); 6.65-6.85 (m, 1H, Ar); 6.90-7.05 (m, 1H, Ar); 7.15-7.63 (m, 9H, Ar); 8.38 (s, 1H, Ar); 10.22-10.43 (m, 2H, NH); 10.56 (br s, 1H, NH) ppm.

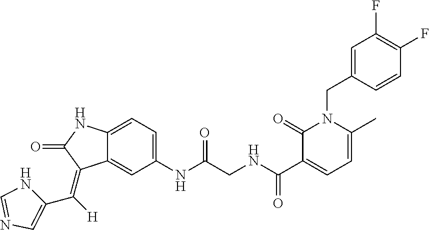 [SMILES: Cc1ccc(C(=O)NCC(=O)Nc2ccc3c(c2)/C(=C/c2cnc[nH]2)C(=O)N3)c(=O)n1Cc1ccc(F)c(F)c1]

### (Z)—N-(2-((3-((1H-imidazol-5-yl)methylene)-2-oxoindolin-5-yl)amino)-2-oxoethyl)-1-(3,4-difluorobenzyl)-6-methyl-2-oxo-1,2-dihydropyridine-3-carboxamide (SST200)

The final product was synthesised following the same procedure described for DD21. (Yield: 45%) C₂₈H₂₂F₂N₆O₄. ¹H-NMR (DMSO): δ 2.43 (s, 3H, CH₃); 4.17 (d, 2H, J=4.8 Hz, CH₂ glycine); 5.40 (s, 2H, CH₂); 6.52 (d, 1H, J=6.8 Hz, Ar); 6.84 (d, 1H, J=8.0 Hz, Ar); 6.98-7.02 (m, 1H, Ar); 7.19 (d, 1H, J=8.0 Hz, Ar); 7.29-7.33 (m, 1H, Ar); 7.39-7.46 (m, 1H, Ar); 7.71-7.74 (m, 2H, Ar); 8.02-8.05 (m, 2H, Ar); 8.30 (d, 1H, J=6.8 Hz, Ar); 10.05 (br s, 2H, NH); 10.92 (brs, 1H, NH) ppm. ¹³C-NMR (DMSO): δ 169.00; 166.96; 163.33; 162.19; 152.42; 150.21; 147.77; 142.74; 139.54; 138.77; 135.75; 133.93; 133.16; 128.01; 124.42; 123.04; 122.80; 120.06; 119.64; 117.79; 117.14; 115.80; 111.15; 109.79; 107.64; 46.40; 43.07; 20.58 ppm. ¹⁹F-NMR: δ: −138.01 (d, 1F, J=24 Hz); −140.50 (d, 1F, J=24 Hz) ppm.

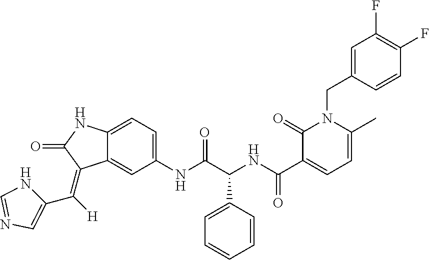 [SMILES: Cc1ccc(C(=O)N[C@@H](C(=O)Nc2ccc3c(c2)/C(=C/c2cnc[nH]2)C(=O)N3)c2ccccc2)c(=O)n1Cc1ccc(F)c(F)c1]

### (R,Z)—N-(2-((3-((1H-imidazol-5-yl)methylene)-2-oxoindolin-5-yl)amino)-2-oxo-1-phenylethyl)-1-(3,4-difluorobenzyl)-6-methyl-2-oxo-1,2-dihydropyridine-3-carboxamide (VI8)

The final product was synthesised following the same procedure described for SA16. (Yield: 37%) C₃₄H₂₆F₂N₆O₄ ¹H-NMR (DMSO): δ 2.41 (s, 3H, CH₃); 5.35-5.50 (m, 2H, CH₂); 5.82 (d, 1H, J=7.2 Hz, Ar); 6.53 (d, 1H, J=7.6 Hz, Ar); 6.83 (d, 1H, J=8.4 Hz, Ar); 6.95-6.97 (m, 1H, Ar); 7.19 (d, 1H, J=8.0 Hz, Ar); 7.31-7.40 (m, 6H, Ar); 7.51-7.53 (m, 2H, Ar); 7.70-7.76 (m, 2H, Ar); 8.00-8.03 (m, 2H, Ar); 8.29 (d, 1H, J=7.2 Hz, Ar); 10.44 (br s, 1H, NH); 10.71 (br d, 1H, J=7.6 Hz, NH); 10.95 (br s, 1H, NH) ppm. ¹³C-NMR (DMSO): δ 169.00; 168.02; 162.56; 162.35; 152.80; 150.11; 147.68; 142.99; 139.63; 138.92; 138.77; 136.00; 133.88; 132.91; 128.66; 128.04; 127.83; 126.80; 124.48; 123.01; 119.88; 119.74; 117.87; 116.82; 115.74; 111.13; 109.84; 107.85; 57.00; 46.45; 20.58 ppm. ¹⁹F-NMR: δ: −137.93 (d, 1F, J=24 Hz); −140.51 (d, 1F, J=24 Hz) ppm.

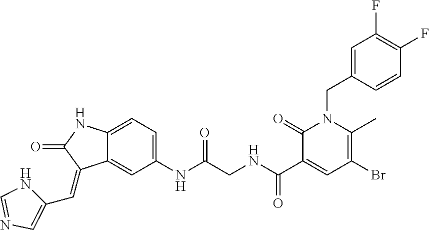 [SMILES: Cc1c(Br)cc(C(=O)NCC(=O)Nc2ccc3c(c2)/C(=C/c2cnc[nH]2)C(=O)N3)c(=O)n1Cc1ccc(F)c(F)c1]

### (Z)—N-(2-((3-((1H-imidazol-5-yl)methylene)-2-oxoindolin-5-yl)amino)-2-oxoethyl)-5-bromo-1-(3,4-difluorobenzyl)-6-methyl-2-oxo-1,2-dihydropyridine-3-carboxamide (VI23)

The final product was synthesised following the same procedure described for SST200. (Yield: 34%) C₂₈H₂₁BrF₂N₆O₄ ¹H-NMR (DMSO): δ 2.55 (s, 3H, CH₃); 4.19 (d, 2H, J=4.8 Hz, CH₂); 5.47 (s, 2H, CH₂); 6.84 (d, 1H, J=8.4 Hz, Ar); 6.95-7.10 (m, 1H, Ar); 7.19 (d, 1H, J=7.6 Hz, Ar); 7.34-7.46 (m, 2H, Ar); 7.71 (s, 1H, Ar) 7.74 (s, 1H, Ar); 8.01-8.05 (m, 2H, Ar); 8.40 (s, 1H, Ar); 9.89-10.05 (br s, 1H, NH); 10.09 (brs, 1H, NH); 10.95 (brs, 1H, NH) ppm. ¹³C-NMR (DMSO): δ 169.05; 166.75; 162.07; 161.15; 150.39; 147.70; 145.36; 139.65; 138.87; 135.82; 133.47; 133.16; 128.07; 124.48; 123.15; 122.92; 120.09; 119.66; 118.45; 117.87; 115.91; 111.17; 109.88; 100.35; 48.19; 43.22; 20.87 ppm. ¹⁹F-NMR: δ: −137.97 (d, 1F, J=24 Hz); −140.39 (d, 1F, J=24 Hz) ppm.

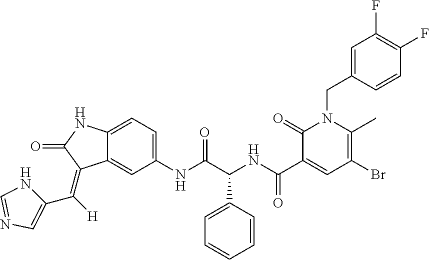 [SMILES: Cc1c(Br)cc(C(=O)N[C@@H](C(=O)Nc2ccc3c(c2)/C(=C/c2cnc[nH]2)C(=O)N3)c2ccccc2)c(=O)n1Cc1ccc(F)c(F)c1]

### (R,Z)—N-(2-((3-((1H-imidazol-5-yl)methylene)-2-oxoindolin-5-yl)amino)-2-oxo-1-phenylethyl)-5-bromo-1-(3,4-difluorobenzyl)-6-methyl-2-oxo-1,2-dihydropyridine-3-carboxamide (VI18)

The final product was synthesised following the same procedure described for VI8. (Yield: 36%) C₃₄H₂₆F₂N₆O₄ ¹H-NMR (DMSO): δ 2.54 (s, 3H, CH₃); 5.42-5.55 (m, 2H, CH₂); 5.82 (d, 1H, J=7.6 Hz, Ar); 6.82 (d, 1H, J=7.6 Hz, Ar); 7.00-7.04 (m, 1H, Ar); 7.19 (d, 1H, J=8.4 Hz, Ar); 7.29-7.43 (m, 5H, Ar); 7.51-7.53 (m, 2H, Ar); 7.71-7.75 (m, 2H, Ar); 7.95-8.02 (m, 2H, Ar); 8.38 (s, 1H, Ar); 10.45 (br s, 1H, NH); 10.61 (br d, 1H, J=7.6 Hz, NH); 10.91 (br s, 1H, NH) ppm. ¹³C-NMR (DMSO): δ 167.81; 161.83; 150.99; 147.55; 148.60; 145.56; 138.58; 136.10; 133.38; 132.80; 128.72; 127.94; 126.82; 124.53; 123.04; 119.78; 118.14; 117.89; 115.82; 111.12; 109.83; 100.51; 57.08; 48.23; 20.88 ppm. ¹⁹F-NMR: δ: −137.91 (d, 1F, J=24 Hz); −140.42 (d, 1F, J=24 Hz) ppm.

Compounds SST201, was prepared according to Scheme 4

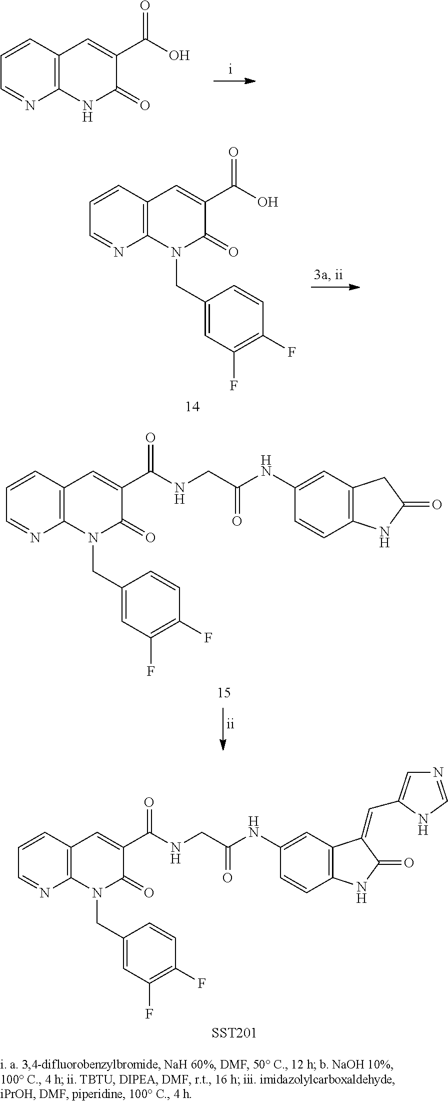 [SMILES: O=C(O)c1cc2cccnc2[nH]c1=O ; O=C(O)c1cc2cccnc2n(Cc2ccc(F)c(F)c2)c1=O ; O=C(CNC(=O)c1cc2cccnc2n(Cc2ccc(F)c(F)c2)c1=O)Nc1ccc2c(c1)CC(=O)N2 ; O=C(CNC(=O)c1cc2cccnc2n(Cc2ccc(F)c(F)c2)c1=O)Nc1ccc2c(c1)/C(=C/c1cnc[nH]1)C(=O)N2 ; "SST201"]

### 1-(3,4-difluorobenzyl)-2-oxo-1,2-dihydro-1,8-naphthyridine-3-carboxylic acid (14)

Compound 14 was synthesized following the same procedure described for compound 5. Yield: 29%. ¹H-NMR (DMSO, 400 Hz) δ 8.92 (s, 1H, Ar); 8.82 (dd, 1H, J=1.6, 4.8 Hz, Ar); 8.55 (dd, 1H, J=2, 8 Hz, Ar); 7.53 (dd, 1H, J=4.8, 7.6 Hz, Ar); 7.38 (m, 2H, Ar), 7.17 (m, 1H, Ar); 5.69 (s, 2H, CH₂).

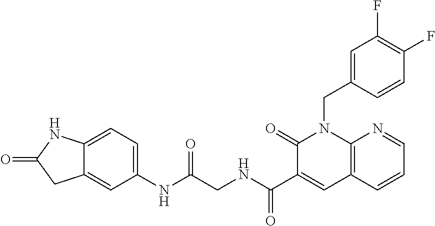 [SMILES: O=C(CNC(=O)c1cc2cccnc2n(Cc2ccc(F)c(F)c2)c1=O)Nc1ccc2c(c1)CC(=O)N2]

### 1-(3,4-difluorobenzyl)-2-oxo-N-(2-oxo-2-((2-oxoindolin-5-yl)amino)ethyl)-1,2-dihydro-1,8-naphthyridine-3-carboxamide (15)

A solution of 1-(3,4-difluorobenzyl)-2-oxo-1,2-dihydro-1,8-naphthyridine-3-carboxylic acid 14 (0.150 g, 0.475 mmol) in anhydrous DMF (18 ml) was stirred in an ice-bath. TBTU (0.152 g, 0.475 mmol) as condensing agent and DIPEA (0.164 ml, 0.95 mmol) as base were added at 0° C. After 30 minutes, 2-oxo-2-((2-oxoindolin-5-yl)amino)ethanaminium 2,2,2-trifluoroacetate (0.151 g, 0.475 mmol) was added and the reaction contents were stirred at 0° C. for 30 minutes and then at room temperature overnight. The mixture was monitoring by TLC. The solvent was evaporated. The solid obtained was triturated in H₂O, filtered and evaporated to give final compound 15 (0,192 g, 0,382 mmol). Yield: 80%. C₂₆H₁₉F₂N₅O₄ ¹H-NMR (DMSO): δ 3.45 (s, 1H, indole); 4.20 (d, 2H, J=5.2 Hz, glycine); 5.70 (s, 2H); 6.74 (d, 1H, J=8.4 Hz); 7.12-7.14 (m, 1H, indole); 7.30-7.46 (m, 3H, Ar); 7.50 (s, 2H, indole); 8.54 (d, 1H, J=6.4 Hz); 8.78 (m, 1H, Ar); 8.96 (s, 1H, Ar); 9.93 (s, 1H, NH); 9.95 (s, 1H, NH); 10.28 (s, 1H, NH) ppm.

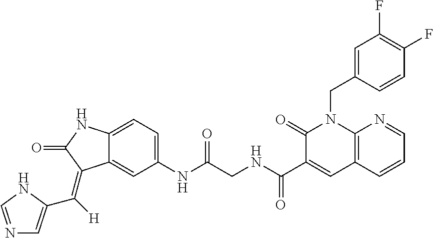 [SMILES: O=C(CNC(=O)c1cc2cccnc2n(Cc2ccc(F)c(F)c2)c1=O)Nc1ccc2c(c1)/C(=C/c1cnc[nH]1)C(=O)N2]

### (Z)—N-(2-((3-((1H-imidazol-5-yl)methylene)-2-oxoindolin-5-yl)amino)-2-oxoethyl)-1-(3,4-difluorobenzyl)-2-oxo-1,2-dihydro-1,8-naphthyridine-3-carboxamide (SST201)

The final compound was synthesized as previously reported for compound SST200. C₃₀H₂₁F₂N₇O₄ ¹H-NMR (DMSO) δ 4.22-4.47 (m, 2H, CH₂ glycine); 5.72 (s, 2H, CH₂); 6.84 (d, 1H, J=7.6 Hz, Ar); 7.16-7.21 (m, 2H, Ar); 7.30-7.54 (m, 4H, Ar); 7.71-7.75 (m, 1H, Ar); 7.91-8.05 (m, 2H, Ar); 8.54-8.56 (m, 1H, Ar); 8.78-8.80 (m, 1H, Ar); 8.99 (s, 1H, Ar); 9.94-10.01 (m, 1H, Ar); 10.68 (br s, 1H, NH); 10.93 (br s, 1H, NH) ppm. ¹⁹F-NMR: δ: −138.70 (dd, 1F, J=24 Hz); −140.86 (d, 1F, J=24 Hz) ppm.

### Example 2

Evaluation of the Compounds of Formula (I)

The activity of the dual inhibitor compounds were assayed utilizing methods known in the art and/or methods presented therein.

The compounds of Formula (I) were tested in the biological test named Kinase-specific Z′-LYTE® assay (Invitrogen Corporation, Life Technologies).

The compounds synthesized were hence subjected to FRET-based Z′-Lyte assay against PDK1/AurA Direct kinase to evaluate the kinase inhibitory activities (Invitrogen).

The IC50 values and percentage of Inhibition are reported in the following table. Data indicate that all the compounds displayed effective results on the inhibition of both PDK1 and AurA kinases. Compounds DD21, DF8, IB35, SA16, displayed the best effects on both PDK1 and Aur A kinases and the potency (IC50) on both enzymes is reported in Table.

| | | IC50 | IC50 |
|---|---|---|---|
| | | PDK1 | AurA |
| | | (nM)% | (nM) or % |
| | | inhibition | inhibition |
| | | PDK1 | AurA |
| Structure | Compound/PM | (10μM) | (10μM) |
| 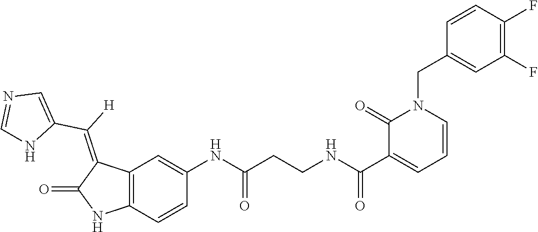 [SMILES: O=C(CCNC(=O)c1cccn(Cc2ccc(F)c(F)c2)c1=O)Nc1ccc2c(c1)/C(=C/c1cnc[nH]1)C(=O)N2] | DD21 (Z) C₂₈H₂₂F₂N₆O₄ PM = 544.52 | 103 | 2470 |
| 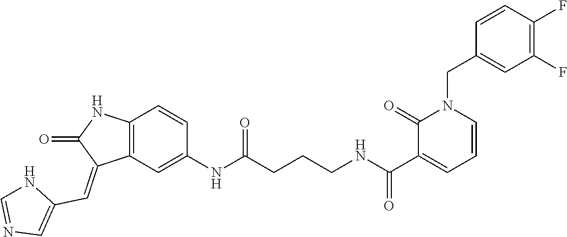 [SMILES: O=C(CCCNC(=O)c1cccn(Cc2ccc(F)c(F)c2)c1=O)Nc1ccc2c(c1)/C(=C/c1cnc[nH]1)C(=O)N2] | DF8 (Z) C₂₉H₂₂F₂N₄O₄S PM = 558.55 | 245 | 611 |
| 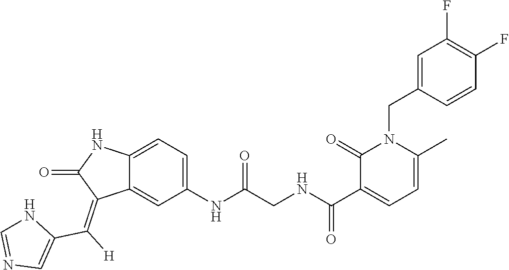 [SMILES: Cc1ccc(C(=O)NCC(=O)Nc2ccc3c(c2)/C(=C/c2cnc[nH]2)C(=O)N3)c(=O)n1Cc1ccc(F)c(F)c1] | SST200 (Z) C₂₈H₂₂F₂N₆O₄ PM = 544,52 | 103 | 193 |
| 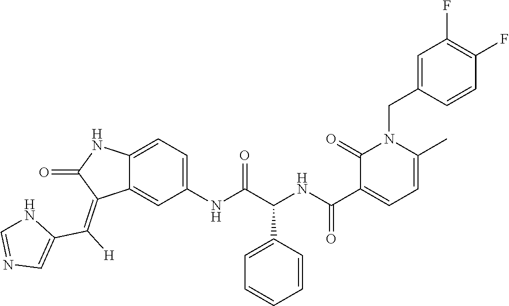 [SMILES: Cc1ccc(C(=O)N[C@@H](C(=O)Nc2ccc3c(c2)/C(=C/c2cnc[nH]2)C(=O)N3)c2ccccc2)c(=O)n1Cc1ccc(F)c(F)c1] | V18 (Z) C₃₄H₂₆F₂N₆O₄ PM = 620,62 | 148 | 69.8 |
| 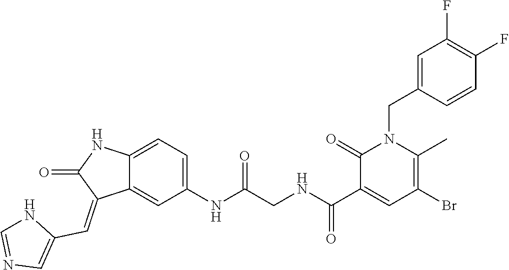 [SMILES: Cc1c(Br)cc(C(=O)NCC(=O)Nc2ccc3c(c2)/C(=C/c2cnc[nH]2)C(=O)N3)c(=O)n1Cc1ccc(F)c(F)c1] | V123 (Z) C₂₈H₂₁BrF₂N₆O₄ PM = 623,41 | 134 | 52% |
| 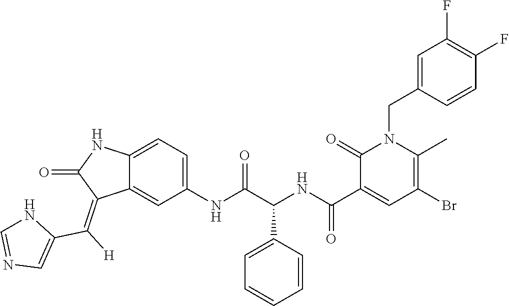 [SMILES: Cc1c(Br)cc(C(=O)N[C@@H](C(=O)Nc2ccc3c(c2)/C(=C/c2cnc[nH]2)C(=O)N3)c2ccccc2)c(=O)n1Cc1ccc(F)c(F)c1] | V118 (Z) C₃₄H₂₅BrF₂N₆O₄ PM = 699,51 | 475 | 118 |
| 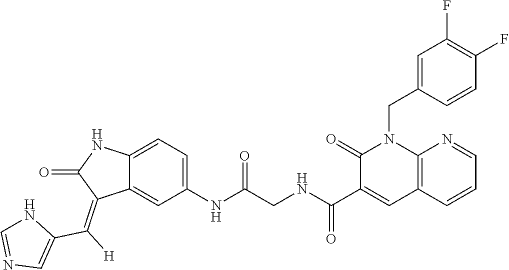 [SMILES: O=C(CNC(=O)c1cc2cccnc2n(Cc2ccc(F)c(F)c2)c1=O)Nc1ccc2c(c1)/C(=C/c1cnc[nH]1)C(=O)N2] | SST201 (Z) C₃₀H₂₁F₂N₇O₄ PM = 581,53 | 35% | 43% |
| 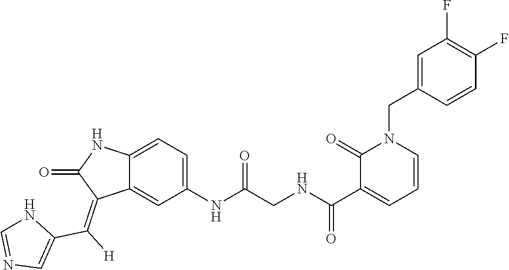 [SMILES: O=C(CNC(=O)c1cccn(Cc2ccc(F)c(F)c2)c1=O)Nc1ccc2c(c1)/C(=C/c1cnc[nH]1)C(=O)N2] | IB35 (Z) C₂₇H₂₀N₆O₄F₂ PM = 530.48 | 112 | 289 |
| 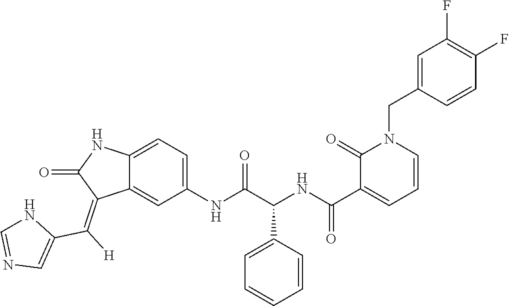 [SMILES: O=C1Nc2ccc(NC(=O)[C@H](NC(=O)c3cccn(Cc4ccc(F)c(F)c4)c3=O)c3ccccc3)cc2/C1=C/c1cnc[nH]1] | SA16 (Z) C₃₃H₂₄F₂N₆O₄ PM = 606.58 | 416 | 35 |

### Example 3 Evaluation of Dual Activity of SA16

SA16 chosen as prototype of this new class of dual PDK1/AurA inhibitors was deeply investigated using as reference drugs MP7 (PDK1 inhibitor) and Alisertib (AurA inhibitor, in clinical trial on different types of tumor.)

The results obtained and described below, showed that SA16 is a dual inhibitor with an antiproliferative activity against GBM cell line which is comparable to that showed by the combination of MP7 and Alisertib. The same trend has been observed in stem cells. Moreover, further investigation on the ability to induce neurosphere differentiation has been performed. Data collected showed that SA16 promotes it and real time PCR analysis showed that the compound is able to promote CSC toward both a neuronal and a glial phenotype

### Example 3a. Effects of MP7 (PDK1 Inhibitor), Alisertib (Aur A Inhibitor), and of their Combined Treatment and SA16 on U87MG

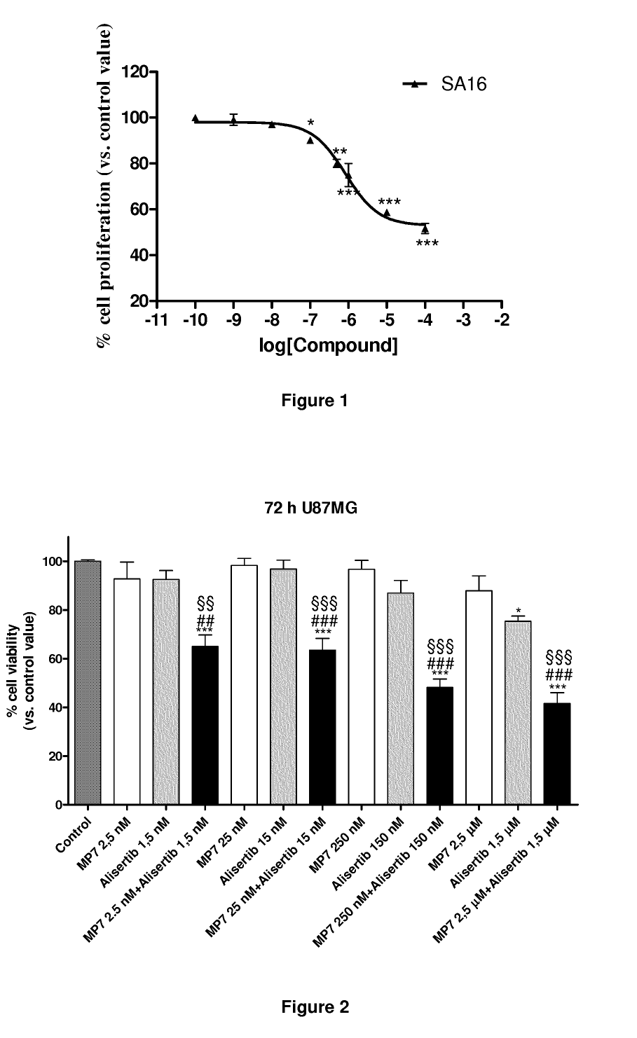

To examine the effects of SA16 on U87MG cell growth/survival, U87MG cells were incubated with different concentrations of the compound SA16 (1 nM-10 μM) for 72 h. SA16 significantly decreased U87MG cell proliferation, in a concentration-dependent manner, with a maximal percentage of inhibition of 49.4±2% as reported in FIG. 1.

In parallel experiments, U87MG proliferation was assessed in the presence of a well-known PDK1 inhibitor MP7, alone or in combination with the Aurora A inhibitor, Alisertib.

After 72 h of cell incubation, MP7 did not show a significant inhibition of cell proliferation (as reported in FIG. 2), consistent with previous report (JBC 2011; 286, 6433-6448). Alisertib alone slightly reduced U87MG cell proliferation; the combined treatment of MP7 and Alisertib showed synergic/additive anti-proliferative effects (FIG. 2), with a maximal percentage of inhibition comparable to that obtained with SA16, thus suggesting that the simultaneous inhibition of PDK1 and AuroraA can be an useful strategy to inhibit GBM cell proliferation.

### Example 3b. Effects of MP7, Alisertib, of their Combined Treatment and SA16 on GBM Stem Cell Viability

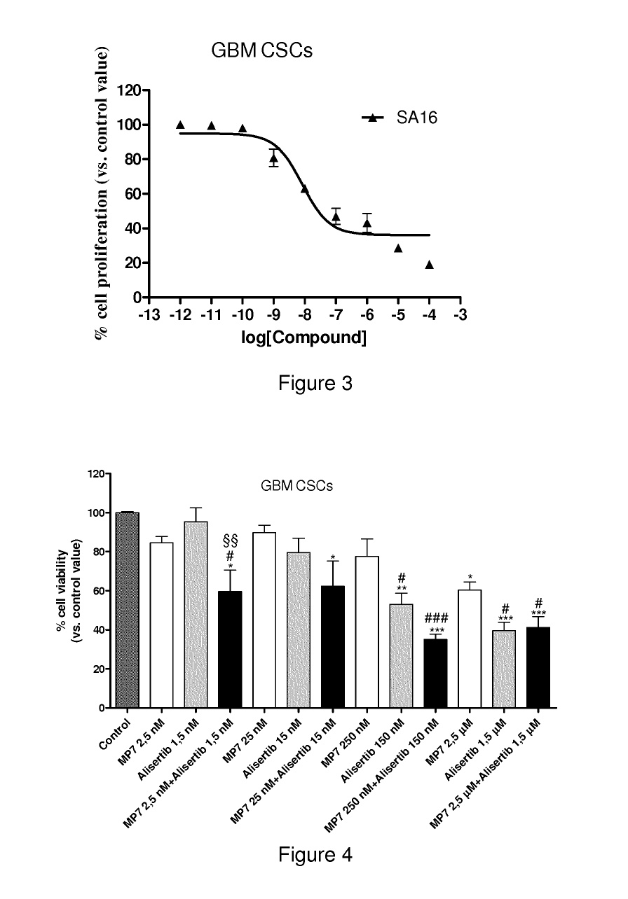

The inventors also examined the effects of SA16 compound on cancer stem cell (CSC) neurospheres obtained from U87MG cells. After seven days of treatment, the compound induced a concentration-dependent inhibition of GSC proliferation, yielding an IC50 value of 8.33±0.78 nM and a maximal percentage of inhibition of 80.0±2.0% (as reported in FIG. 3).

Consistent with the data obtained in U87MG cells, MP7 (PDK1 inhibitor chosen as reference drug) administered alone showed slight effects on U87MG proliferation. Alisertib anti-proliferative effects on CSCs, in a concentration-dependent manner, thus confirming that Aurora A inhibition shows a higher efficacy in neurosphere cells with respect to standard monolayer GBM cells (Cancer Chemother Pharmacol 2014; 73:983-990).

The combination of the two compounds decreased CSC proliferation, in a concentration-dependent manner, with percentages of inhibition significantly higher with respect to those obtained in single-treated CSCs. The maximal effects of the co-treatment protocol were even lower than those obtained with the dual target compound SA16 (as reported in FIG. 4).

### Example 3c. Effects of SA16, MP7, Alisertib and of their Combined Treatment on CSC Morpholoqy and Differentiation

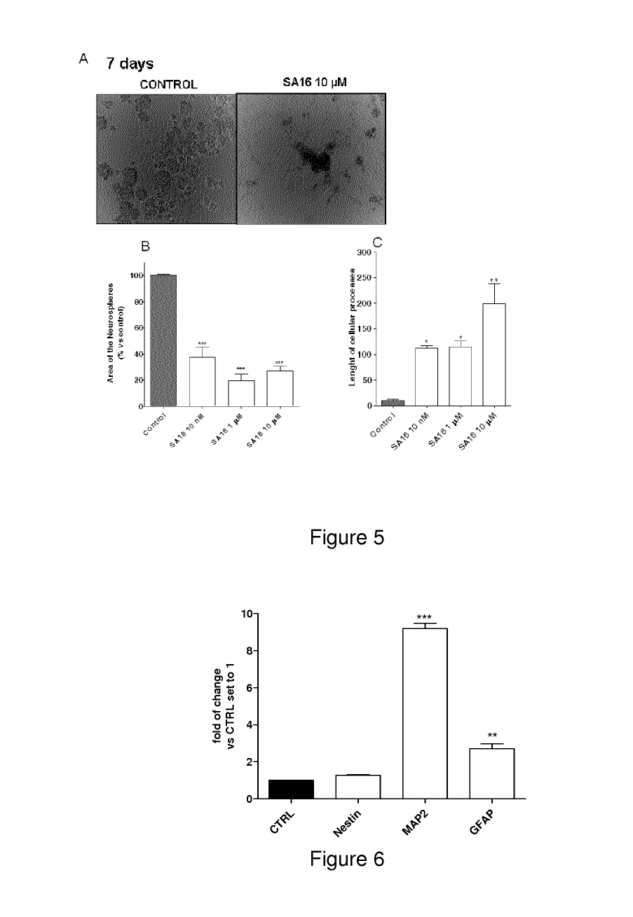

The effects of SA16 on morphology of neurospheres were evaluated by quantifying the area occupied by the cells in culture plates, as well as the outgrowth of cellular processes. When neurospheres were incubated with SA16 for seven days at different concentrations (10 nM, 1 μM, 10 μM), an almost complete reduction in the area occupied by the neurospheres was noticed (FIGS. 5A and B), and the cells showed prominent outgrowth of processes (FIGS. 5A and C). The differentiating effects of SA16 were confirmed by real time PCR analysis (FIG. 6), which showed that the compound is able to promote CSC toward both a neuronal and a glial phenotype.

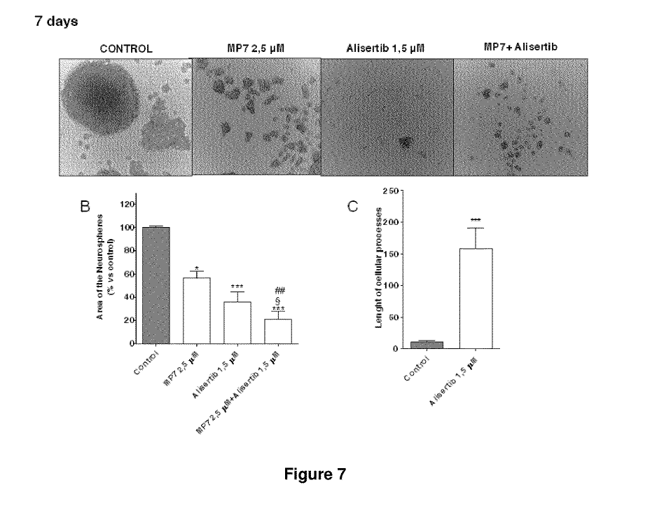

The inventors then evaluated the effects of MP7, Alisertib and of their combination on CSC differentiation. The cells were incubated with 2.5 PIM MP7, alone or in combination with 1.5 μM Alisertib for seven days. The two compound each administered alone led to a reduction in the area occupied by the neurospheres (FIGS. 7A and B); these effects were evident particularly in cells treated with Alisertib; moreover, in this case, CSC showed modest but significant outgrowth of cellular processes (FIGS. 7A and C); thus confirming that AuroraA inhibition induces CSC differentiation (Cancer Chemother Pharmacol 2014; 73:983-990).

When MP7 and Alisertib were combined together, a synergic/additive effect on the reduction of neurospheres' area was evidenced (FIG. 7), thus suggesting that the combined inhibition of PDK1 and AuroraA could be an useful strategy to inhibit CSC proliferation and induce differentiation. Altogether the results obtained by the combination of MP7 with Alisertib indicate that the inhibition of CSC proliferation and the capability to induce differentiation are lower than that induce by the dual inhibitor SA16 alone.

1. A method for treating pathologies requiring the use of a dual inhibitor of PDK1/AurA enzymes, said method comprising administering a 2-oxo-1,2-dihydropyridine-3-carboxamide compound of Formula (I) 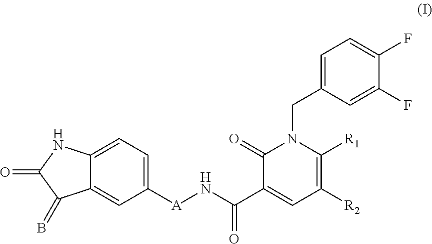
or a pharmaceutical salt thereof
wherein
B is CH-D, where D is imidazolyl; and
A is selected from the group consisting of (—NH—CO—CH₂—), (—NH—CO—CH₂—CH₂), (—NH—CO—CH(Ph)-) and (—NH—CO—CH₂—CH₂—CH₂)
R₁ is H or CH₃, R₂ is H or Br or R₁ and R₂ taken together form the group —(N═CH—CH═CH)—
with the proviso that
when A is selected from (—NH—CO—CH₂—CH₂) and (—NH—CO—CH₂—CH₂—CH₂), then R₁ and R₂ are H.

2. The method of claim 1, wherein A is (—NH—CO—CH₂—) or (—NH—CO—CH(Ph)-).

3. The method of claim 1, wherein A is (—NH—CO—CH(Ph)-).

4. The method of claim 3, wherein R₁ is CH₃ and R2 is H.

5. The method of claim 3, wherein R₁ is CH₃ and R2 is Br.

6. The method of claim 3, wherein R₁ and R₂ are H.

7. The method of claim 1, wherein A is (—NH—CO—CH₂—CH₂).

8. The method of claim 1, wherein D is 1H-imidazol-5-yl.

9. The method of claim 1, wherein the compound is selected from the group consisting of (Z)—N-(4-((3-((1H-imidazol-5-yl)methylene)-2-oxoindolin-5-yl)amino)-4-oxobutyl)-1-(3,4-difluorobenzyl)-2-oxo-1,2-dihydropyridine-3-carboxamide(DF8), (Z)—N-(2-((3-((1H-imidazol-5-yl)methylene)-2-oxoindolin-5-yl)amino)-2-oxoethyl)-1-(3,4-difluorobenzyl)-2-oxo-1,2-dihydro pyridine-3-carboxamide(IB35), (Z)—(R)—N-(2-((3-((1H-imidazol-5-yl)methylene)-2-oxoindolin-5-yl)amino)-2-oxo-1-phenyl ethyl)-1-(3,4-difluorobenzyl)-2-oxo-1,2-dihydropyridine-3-carboxamide (SA16), (Z)—N-(3-((3-((1H-imidazol-5-yl)methyl ene)-2-oxoindolin-5-yl)amino)-3-oxopropyl)-1-(3,4-difluorobenzyl)-2-oxo-1,2-dihydropyridine-3-carboxamide (DD21), (Z)—N-(2-((3-((1H-imidazol-5-yl)methylene)-2-oxoindolin-5-yl)amino)-2-oxoethyl)-1-(3,4-difluorobenzyl)-6-methyl-2-oxo-1,2-dihydropyridine-3-carboxamide (SST200), (R,Z)—N-(2-((3-((1H-imidazol-5-yl)methylene)-2-oxoindolin-5-yl)amino)-2-oxo-1-phenylethyl)-1-(3,4-difluorobenzyl)-6-methyl-2-oxo-1,2-dihydropyridine-3-carboxamide (VI8), (Z)—N-(2-((3-((1H-imidazol-5-yl)methyl ene)-2-oxoindolin-5-yl)amino)-2-oxoethyl)-5-bromo-1-(3,4-difluorobenzyl)-6-methyl-2-oxo-1,2-dihydropyridine-3-carboxamide (VI23). (R,Z)—N-(2-((3-((1H-imidazol-5-yl)methylene)-2-oxoindolin-5-yl)amino)-2-oxo-1-phenylethyl)-5-bromo-1-(3,4-difluorobenzyl)-6-methyl-2-oxo-1,2-dihydropyridine-3-carboxamide (VI18) and (Z)—N-(2-((3-((1H-imidazol-5-yl)methylene)-2-oxoindolin-5-yl)amino)-2-oxoethyl)-1-(3,4-difluorobenzyl)-2-oxo-1,2-dihydro-1,8-naphthyridine-3-carboxamide (SST201).

10. The method of claim 9, wherein the compound is selected from the group (Z)—N-(4-((3-((1H-imidazol-5-yl)methylene)-2-oxoindolin-5-yl)amino)-4-oxobutyl)-1-(3,4-difluorobenzyl)-2-oxo-1,2-dihydropyridine-3-carboxamide(DF8), (Z)—N-(2-((3-((1H-imidazol-5-yl)methylene)-2-oxoindolin-5-yl)amino)-2-oxoethyl)-1-(3,4-difluorobenzyl)-2-oxo-1,2-dihydro pyridine-3-carboxamide(IB35), (Z)—(R)—N-(2-((3-((1H-imidazol-5-yl)methylene)-2-oxoindolin-5-yl)amino)-2-oxo-1-phenylethyl)-1-(3,4-difluorobenzyl)-2-oxo-1,2-dihydropyridine-3-carboxamide (SA16), (Z)—N-(2-((3-((1H-imidazol-5-yl)methylene)-2-oxoindolin-5-yl)amino)-2-oxoethyl)-1-(3,4-difluorobenzyl)-6-methyl-2-oxo-1,2-dihydropyridine-3-carboxamide (SST200), (R,Z)—N-(2-((3-((1H-imidazol-5-yl)methyl ene)-2-oxoindolin-5-yl)amino)-2-oxo-1-phenyl ethyl)-1-(3,4-difluorobenzyl)-6-methyl-2-oxo-1,2-dihydropyridine-3-carboxamide (VI8), and (R,Z)—N-(2-((3-((1H-imidazol-5-yl)methyl ene)-2-oxoindolin-5-yl)amino)-2-oxo-1-phenyl ethyl)-5-bromo-1-(3,4-difluorobenzyl)-6-methyl-2-oxo-1,2-dihydropyridine-3-carboxamide (VI18).

11. The method of claim 10, wherein the compound is selected from the group consisting of (Z)—(R)—N-(2-((3-((1H-imidazol-5-yl)methylene)-2-oxoindolin-5-yl)amino)-2-oxo-1-phenylethyl)-1-(3,4-difluorobenzyl)-2-oxo-1,2-dihydropyridine-3-carboxamide (SA16), (Z)—N-(2-((3-((1H-imidazol-5-yl)methylene)-2-oxoindolin-5-yl)amino)-2-oxoethyl)-1-(3,4-difluorobenzyl)-6-methyl-2-oxo-1,2-dihydropyridine-3-carboxamide (SST200) and (R,Z)—N-(2-((3-((1H-imidazol-5-yl)methylene)-2-oxoindolin-5-yl)amino)-2-oxo-1-phenylethyl)-1-(3,4-difluorobenzyl)-6-methyl-2-oxo-1,2-dihydropyridine-3-carboxamide (VI8).

12. A 2-oxo-1,2-dihydropyridine-3-carboxamide compound of Formula (I) 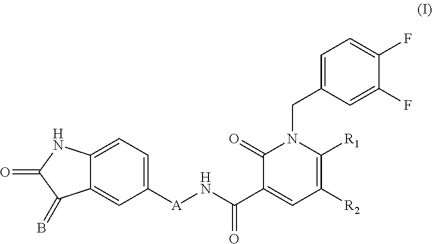
or a pharmaceutical salt thereof
wherein
B is CH-D, where D is imidazolyl; and
A is selected from the group consisting of (—NH—CO—CH₂—), (—NH—CO—CH(Ph)-) and (—NH—CO—CH₂—CH₂—CH₂)
R₁ is H or CH₃, R₂ is H or Br or R₁ and R₂ together represent the group —(N═CH—CH═C)—
with the proviso that
when A is (—NH—CO—CH₂—CH₂—CH₂) then R₁ and R₂ are H and
when A is (—NH—CO—CH₂—) or (—NH—CO—CH(Ph)-), then R₁ and R₂ are not simultaneously H.

13. The compound of claim 12, wherein D is imidazolyl, preferably 1H-imidazol-5-yl.

14. The compound of claim 12, wherein A is (—NH—CO—CH(Ph)-).

15. The compound of claim 14, wherein R₁ is CH₃ and R₂ is H.

16. The compound of claim 14, wherein R₁ is CH₃ and R₂ is Br.

17. The compound of claim 12, said compound being selected from the group consisting of (Z)—N-(4-((3-((1H-imidazol-5-yl)methylene)-2-oxoindolin-5-yl)amino)-4-oxobutyl)-1-(3,4-difluorobenzyl)-2-oxo-1,2-dihydropyridine-3-carboxamide(DF8), (Z)—N-(2-((3-((1H-imidazol-5-yl)methylene)-2-oxoindolin-5-yl)amino)-2-oxoethyl)-1-(3,4-difluorobenzyl)-6-methyl-2-oxo-1,2-dihydropyridine-3-carboxamide (SST200), (R,Z)—N-(2-((3-((1H-imidazol-5-yl)methylene)-2-oxoindolin-5-yl)amino)-2-oxo-1-phenylethyl)-1-(3,4-difluorobenzyl)-6-methyl-2-oxo-1,2-dihydropyridine-3-carboxamide (VI8), (Z)—N-(2-((3-((1H-imidazol-5-yl)methylene)-2-oxoindolin-5-yl)amino)-2-oxoethyl)-5-bromo-1-(3,4-difluorobenzyl)-6-methyl-2-oxo-1,2-dihydropyridine-3-carboxamide (VI23), (R,Z)—N-(2-((3-((1H-imidazol-5-yl)methylene)-2-oxoindolin-5-yl)amino)-2-oxo-1-phenyl ethyl)-5-bromo-1-(3,4-difluorobenzyl)-6-methyl-2-oxo-1,2-dihydropyridine-3-carboxamide (VI18) and (Z)—N-(2-((3-((1H-imidazol-5-yl)methylene)-2-oxoindolin-5-yl)amino)-2-oxoethyl)-1-(3,4-difluorobenzyl)-2-oxo-1,2-dihydro-1,8-naphthyridine-3-carboxamide (SST201).

18. The compound of claim 17, wherein the compound is (Z)—N-(2-((3-((1H-imidazol-5-yl)methylene)-2-oxoindolin-5-yl)amino)-2-oxoethyl)-1-(3,4-difluorobenzyl)-6-methyl-2-oxo-1,2-dihydropyridine-3-carboxamide (SST200) or (R,Z)—N-(2-((3-((1H-imidazol-5-yl)methylene)-2-oxoindolin-5-yl)amino)-2-oxo-1-phenylethyl)-1-(3,4-difluorobenzyl)-6-methyl-2-oxo-1,2-dihydropyridine-3-carboxamide (VI8).

19. (canceled)

20. A pharmaceutical composition comprising: the 2-oxo-1,2-dihydropyridine-3-carboxamide compound of claim 12 and a pharmaceutically acceptable carrier.

21. A method for treating a pathology requiring the use of a dual inhibitor of PDK1/AurA enzymes, said method comprising administering the 2-oxo-1,2-dihydropyridine-3-carboxamide compound of anyone of claim 12 to a subject having said pathology.

22. The method of claim 21, wherein the pathology is selected from the group consisting of tumours, primary colorectal carcinoma, gliomas, breast, ovarian, pancreatic cancer, hematologic malignancies, multiple myeloma, Non-Hodgkin lymphoma, chronic lymphocytic leukemia, glioblastoma, neurodegenerative diseases, Alzheimer's disease, Parkinson's disease, Huntington's disease, cardiovascular diseases, diabetes.

23. The method of claim 22, wherein the pathology is a cancer, preferably glioblastoma (GBM).

24. A pharmaceutical combination comprising at least one PDK1 inhibitor and at least one AurA inhibitor.

25. The pharmaceutical combination according to claim 24, wherein the at least one PDK1 inhibitor is MP7 and the at least one AurA inhibitor is Alisertib.

26. A method for treating a pathology requiring the use of a dual inhibitor of PDK1/AurA enzymes, said method comprising administering the pharmaceutical combination according to claim 24 to a subject having said pathology.
